(19) United States
(12) Patent Application Publication
(10) Pub. No.: US 2022/0068462 A1
(43) Pub. Date: Mar. 3, 2022

(54) Artificial Memory for use in Cognitive Behavioral Therapy Chatbot

(71) Applicant: doc.ai, Inc., Palo Alto, CA (US)

(72) Inventors: Eric William DOLAN, Woodbridge (CA); Joel Thomas KAARDAL, San Mateo, CA (US); Jeremy Fine HARTMAN, Menlo Park, CA (US); Satish Srinivasan BHAT, Fremont, CA (US); Srivatsa Akshay SHARMA, Palo Alto, CA (US); Walter Adolf DE BROUWER, Los Altos, CA (US)

(73) Assignee: doc.ai, Inc., Palo Alto, CA (US)

(21) Appl. No.: 17/006,743

(22) Filed: Aug. 28, 2020

(51) Int. Cl. G16H 20/70; H04L 12/58; G10L 15/26; G10L 25/63; G06F 40/295

(52) U.S. Cl. CPC G16H 20/70; H04L 51/02; G06F 40/295; G10L 25/63; G10L 15/26

(57) ABSTRACT

The technology disclosed relates to a system and method for remembering content received from a patient for future use and understood by computerized natural language processing during a chatbot therapy session. The system includes a graph of entities and relationships in an artificial memory graph data structure. The entity nodes in the graph represent entities that are related to a patient root node representing the patient. The entity nodes are connected to patient root node by one or more relationship edges that have named roles. The entity nodes and/or relationship edges can be slotted for at least a distinguishing name, a patient sentiment towards the entity and an entity state.

### FIELD OF THE TECHNOLOGY DISCLOSED

The technology disclosed relates to the use of artificial memory for conducting a mental health therapy session by a chatbot that can support tangential conversations.

### BACKGROUND

The subject matter discussed in this section should not be assumed to be prior art merely as a result of its mention in this section. Similarly, a problem mentioned in this section or associated with the subject matter provided as background should not be assumed to have been previously recognized in the prior art. The subject matter in this section merely represents different approaches, which in and of themselves can also correspond to implementations of the claimed technology.

Cognitive behavioral therapy, CBT, has been successfully used to treat mental health issues in diverse sets of populations with a wide range of disorders and problems. Access to treatment is still an issue due to limited availability of trained therapists and high treatment costs. A CBT therapy module for a mental health disorder can consist of a series of sessions that are conducted weekly or biweekly. Computer-based CBT therapy chatbots can be developed to address the challenges regarding availability of trained therapists and the cost of the treatment. However, there are several technical challenges in achieving the goal of providing computer-based CBT therapy treatment. The therapists are trained to build a therapeutic relationship with patients over time. They learn about patient's relationships to other people in their personal and professional lives and patient's sentiments towards those people. The therapist then uses the information collected over a series of sessions to build a relationship with the patient.

The CBT therapy sessions are linearly structured with predefined goals for each session. However, the patient is not always up to the task during a session. The patient can bring up other thoughts during the conversation with the therapist. Some of these thoughts may be more important for the patient than the scheduled session. Therapists are trained to detect when to shift topics from the structured steps of the session and go on a tangential conversation with the patient. The therapist brings back the conversation to the main therapy session after concluding the tangential conversation. A therapist is also trained to determine the length of the tangential conversation. A computer is limited in its ability to detect when to shift from the linear structure of the conversation and start a tangential conversation. Without this capability, the therapy sessions can become mechanical and the patient can lose interest in the session.

An opportunity arises to develop a mental health chatbot that can not only handle tangential conversations but also build a therapeutic relationship with the patient over the course of multiple CBT therapy sessions.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The color drawings also may be available in PAIR via the Supplemental Content tab.

FIG. 1 illustrates a block diagram of an example environment in which a mental health chatbot can provide CBT therapy treatment to patients.

FIG. 2A is a block diagram of components of a mental health chatbot.

FIG. 2B presents examples of dependency parsing and entity extraction from utterances of patients.

FIGS. 3A and 3B present an example CBT therapy script for staying happy while isolated.

FIGS. 4A and 4B present an example CBT therapy script for structured problem solving.

FIG. 5A presents an example of artificial memory graph data structure for storing entities related to patients.

FIG. 5B presents an example of global knowledge graph data structure for storing public data related to patients.

FIGS. 6A and 6B present examples of hypernym and hyponym relationships and a relationship ontology data structure.

FIGS. 7A and 7B present example conversations of chatbot with patient that include queries to artificial memory graph data structure to extract entities and relationships.

FIG. 8 presents examples of structured and unstructured conversations and detecting intent of the patient to start a tangential conversation.

FIG. 9 presents linearly structured conversation and conversations of chatbot with patient that include tangential branches.

FIG. 10 is an example conversation of the chatbot with patient that includes accessing artificial memory and starting a tangential conversation.

FIG. 11 is an example conversation in which the chatbot provides the patient with multiple wellness tools from which the patient can select one to start a tangential conversation.

FIG. 12 is a block diagram of a computer system that can be used to implement the mental health chatbot of FIG. 1.

### DETAILED DESCRIPTION

The following discussion is presented to enable any person skilled in the art to make and use the technology disclosed, and is provided in the context of a particular application and its requirements. Various modifications to the disclosed implementations will be readily apparent to those skilled in the art, and the general principles defined herein may be applied to other implementations and applications without departing from the spirit and scope of the technology disclosed. Thus, the technology disclosed is not intended to be limited to the implementations shown, but is to be accorded the widest scope consistent with the principles and features disclosed herein.

### INTRODUCTION

The next generation of Serenity mental health chatbot app will come closer to passing Turing's test (en.wikipedia.org/wiki/Turing test), applied to a cognitive behavioral therapy chat, because it will accommodate tangential conversation and be able to resolve ambiguous relationship references used by a patient.

Cognitive behavioral therapy, CBT, is structured as a series of modules that address life events and situations. For instance, dealing with grief from death of a family pet or stress from starting a new job. As a patient engages in multiple CBT sessions, they will not always be on task. They will launch tangential conversations or digressions, some of which are more important to the patient than the scheduled module. A therapist naturally handles tangential conversations and recognizes when to shift topics. Some natural language processing mechanisms, such as topic analysis and intent classification, can be used to recognize when the subject of conversation changes. The technology disclosed extends prior mechanisms to therapy, enabling the mental health chatbot to progress through a CBT module and still entertain tangential conversations for brief or extended periods. It better recognizes from the patient's utterances a patient's intent to digress and better accommodates the patient, by not losing track of the CBT goal and state of progress.

The chatbot can recognize events in patient utterances and infer patient sentiments to empathize and even to propose targeted therapy exercises. Consider the example of a session in a multi-session CBT module for dealing with negative thoughts that is interrupted by the patient describing an argument with their spouse. The chatbot can recognize from the patient's utterance that the patient wants to talk about this event which may be tangential to the linearly structured steps of the current therapy session. The chatbot can detect from a patient's utterance that the event involves the patient's spouse. The chatbot can further infer from the event that the patient has a negative sentiment towards their spouse in the utterance. The chatbot can present a specific therapy exercise or coping module to the patient to deal with their sentiment towards their spouse. For example, in this case, the chatbot can invite the patient to “healthy disagreements with your partner” therapy exercise. If the patient accepts the invitation, a last completed step of the CBT therapy session is bookmarked, the chatbot starts a tangential conversation with the patient to complete the proposed therapy exercise. The chatbot resumes the CBT therapy session from the bookmarked position after concluding the tangential conversation. The tangential conversations can also be initiated by the patient by specifically asking the chatbot to talk about a particular event.

The technology disclosed builds the therapeutic relationship from session to session, both by progression through sessions in a module and across modules and by remembering what it has heard. In ordinary conversation, we often use pronouns and relational terms to refer to important aspects of our life. For instance, we say my dog, my sister, my wife, or where I grew up. A therapist remembers that my dog is Lulu, my sister is Eileen, my wife is Ginny, and Chicago is where I grew up. The technology disclosed includes a patient-specific artificial memory and triggers for collecting information that resolves relationship entities or named roles remembers state information such as whether the dog is dead or alive, and remembers sentiment information such as how well I am getting along with my sister. It includes query mechanisms for resolving pronouns, retrieving state and sentiment information, and using retrieved memories in the therapeutic dialogue.

The technology disclosed captures entities and their relationships to the patient from conversations with the patient. Examples of entities include people, pets, places, or other animate or inanimate things. The patient-specific artificial memory is a graph data structure that can store entity nodes and relationship edges. The entity nodes are connected to the patient root by relationship edges. The entity nodes can be slotted for a distinguishing name, a patient sentiment towards the entity and an entity state. The entity nodes can include additional slots for storing other attributes for the entities such as a type attribute indicating whether the entity is a person or a pet, a time stamp attribute for first and last mentions of the entity by the patient, a religion attribute, an education attribute, and a workplace attribute, etc.

The patient root and the entity nodes can be connected by multiple edges that can have different relationship names or roles. For example, the patient root can be connected to a wife entity node with two relationship edges: a spouse relationship edge and a wife relationship edge. To keep track of alternative expressions of relationships, the technology includes a relationship hierarchy of hypernym (e.g., spouse) and hyponym (e.g., wife, husband) relationships. As the chatbot collects more information about the entities over the course of multiple CBT therapy sessions, new edges are added between the entity nodes and the patient root based on information from the patient. The relationship edges can also be slotted to store a relationship name or a named role, and other attributes such as patient's sentiment towards the entity, etc. Examples of named roles include sibling, sister, brother, parent, mother, father, friend, etc.

These innovations and improvements to the Serenity mental health chatbot make it harder to distinguish user interactions with Serenity during CBT modules from chatting with a human therapist. The flexibility to engage in tangential conversation, then, eventually, return to the module at hand makes the interaction less mechanical. Building on what the chatbot can remember from the patient's ongoing description of relationships, including resolution of hypernyms and memory of sentiments towards people, places and things, makes a multi-session chat with Serenity feel like working with a CBT therapist.

### Environment

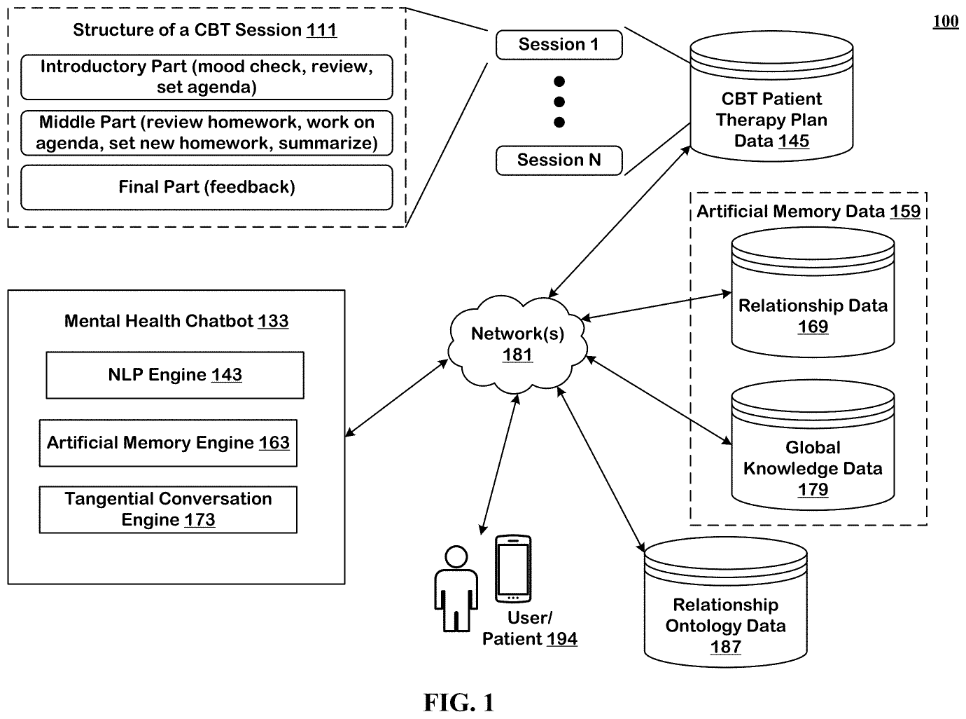

We describe a system for a mental health chatbot that can conduct cognitive behavioral therapy sessions with patients by acting as a therapist. Cognitive Behavioral Therapy (CBT) or Cognitive Therapy is a type of psychotherapy. In general, psychotherapy refers to treatment of mental disorders by psychological rather than medical means. When conducting CBT therapy sessions, the system includes natural language processing logic to remember content received from a patient for future chatbot therapy sessions. The system can make use of the remembered content during a therapy session from earlier interactions with the patient in a series of sessions. The system also includes logic to detect when to diverge to a tangential conversation from linearly structured steps of a CBT session and when to merge back to the flow of linearly structured steps of the session from the tangential conversation in the therapy session. The system is described with reference to FIG. 1 showing an architectural level schematic of a system in accordance with an implementation. Because FIG. 1 is an architectural diagram, certain details are intentionally omitted to improve the clarity of the description. The discussion of FIG. 1 is organized as follows. First, the elements of the figure are described, followed by their interconnection. Then, the use of the elements in the system is described in greater detail.

FIG. 1 includes the system 100. This paragraph names labeled parts of system 100. The figure includes a mental health chatbot 133, a user (or a patient) 194, a CBT patient therapy plan database 145, a high-level structure of a CBT session 111, an artificial memory database 159, and a relationship ontology database 187. The mental health chatbot 133 can comprise of a natural language processing (NLP) engine 143, artificial memory engine 163, and a tangential conversation engine 173. The artificial memory database can comprise of a relationship database 169 and a global knowledge database 179.

CBT patient therapy plan database 145 can comprise of one or more therapy modules that address life events and situations in the patient's life. For example, one CBT module can treat the patient to deal with grief from death of a family pet, a second CBT module can treat the patient to deal with stress of a new job, etc. A CBT module can have up to N sessions. In most instances the treatment in a CBT module span from 5 therapy sessions up to about 20 therapy sessions. A session can have a duration of 30 to 60 minutes.

A network(s) 181 couples the mental health chatbot 133, the patient 194, the CBT patient therapy plan database 145 containing up to N therapy sessions for each therapy module, an artificial memory database 159, and a relationship ontology database 187.

The treatment in CBT is based on cognitive formulation, the beliefs and behavioral strategies that characterize a specific disorder (Alford & Beck, 1997). Treatment is also based on conceptualization and understanding of specific beliefs and patterns of behavior of individual patients. The therapist seeks in a variety of ways to produce cognitive change—modification in the patient's thinking and belief system—to bring about enduring emotional and behavioral change. The CBT is used to manage a variety of conditions in the categories of psychiatric disorders, psychological problems, and medical problems with psychological components. Examples of psychiatric disorders include depressive disorder, panic disorder, substance abuse, health anxiety, etc. Examples of psychological problems include couple problems, family problems, grief, anger and hostility, etc. Examples of medical problems with psychological components include insomnia, migraine headaches, chronic back pain, etc.

CBT sessions are structured. No matter what the diagnosis or stage of treatment, following a certain structure in each session improves efficiency and effectiveness. The structure of a CBT session 111 can include an introductory part, a middle part, and a final part. The structure of a CBT session is illustrated by the element 111 of system 100 in FIG. 1. The introductory part can include performing a mood check of the patient, brief review of the week, and collaboratively setting an agenda for the session. For effective therapy, the current session in the module is linked to the previous session using a bridge. The summary of the previous week can include important issues addressed during the previous session. Its purpose is to help patient remember what was important in the previous session and ensures that the therapist and patient are on the same page. The middle part can include reviewing the homework, discussing problems on the agenda, setting new homework, and summarizing the session. The final part can include eliciting feedback from the patient. Following this format makes the process of therapy more understandable to patients and increases the likelihood that they will be able to self-therapy after completion of the therapy module.

For effective and successful treatment, the therapist builds a therapeutic relationship with the patient from session to session in a therapy module and across multiple therapy modules. The technology disclosed includes patient specific artificial memory (also referred to as Mercer memory) database 159 for storing information collected from patient's utterances. The system includes logic to resolve pronouns and hypernyms in a patient's utterances and remember the state information such as whether a pet (or another entity) mentioned in patient's utterance is dead or alive. The system also includes logic to remember sentiment information of the patient towards other entities such as her spouse, her boss, her sibling, etc. The system includes triggers to collect such information from utterances of the patient during a session. The system also includes logic to resolve the pronouns and ambiguities to link the information to the correct entity. For example, during a session, if the patient utters, “I had a long discussion with my daughter”. The system can query the artificial memory data structure for the patient's daughter and find out that the patient has two daughters, then the system can respond to the patient “Is this Mary or Jessica?” to disambiguate and link the information in the following conversation to the correct entity.

The artificial memory database can include two separate databases or data structures, a relationship database 169 and a global knowledge database 179. The relationship database includes entities, relationship of the patient to the entities, and the relationship between entities as mentioned by the patient. The entities can represent people, pets, places, and other animate or inanimate things. The system can maintain the patient specific artificial memory database as a graph data structure consisting of entity nodes representing entities that are related to a patient root node representing the patient. The entity nodes are connected to the patient root by one or more relationship edges that have named roles. The entity nodes and relationship edges can be slotted for distinguishing name, a patient sentiment towards the entity, entity state and other attributes.

The technology disclosed includes maintaining a relationship ontology (or relationship hierarchy) that describes alternative relationship edges used to connect the patient root node to a particular entity node. The relationship ontology can be structured as hypernym and hyponym relationships. In linguistics, a hyponym is a word or phrase whose meaning is included within that of another word, its hypernym. In simple words, a hyponym is in a type-of relationship with its hypernym. For example, brother, sister are hyponyms of sibling (their hypernym), and husband, wife are hyponyms of spouse (their hypernym). The technology disclosed includes the logic to add more edges between the entity nodes as more information is received from the patient about her relationships to other entities. An entity node may be connected to the patient root node with a “sibling” edge before the chatbot receives further details from the patient about her relationship to the entity indicating that the entity is her sister. The system can then add another relationship edge indicating that the entity is patient's sister. The technology disclosed can maintain a relationship ontology database 187 that can include a hierarchy of hypernyms and hyponyms.

The technology disclosed includes logic to use relationship ontology to resolve complex relationships and represent these as primitive relationships. For example, if patient mentions her “aunt” in an utterance. The entity node representing the aunt is connected to the patient root via relationship ( )-[aunt]->( ). The system can also resolve this complex relationship into a primitive relationship which can include multiple hops from the patient root to the same entity node. For example, the above relationship can also be represented as ( )-[parent]->( )-[sister]->( ). We reach the same entity node after this resolution but the system uses multiple primitive relationships (parent and sister) as opposed to a single complex relationship (aunt).

The global knowledge database 179 can be shared across multiple patients. This database can include information about places, things, events, or other entities that a patient mentions in her utterances. For example, a patient can mention that she was born in Chicago and spent her childhood over there. The system can then link the patient root node in relationship database 169 to a knowledge base such as WikiData (wikidata.org) record for Chicago. In one implementation, the entities in global knowledge graph can also be represented as a graph data structure with entities and relationships. The global knowledge database 179 is not patient specific. The patient root node can be connected to one or more entity nodes (or records) in the global knowledge database.

The mental health chatbot 133 can build on what the chatbot can remember from the patient's ongoing description of relationships including resolution of hypernyms and memory of sentiments towards people, pets, places, and things. The chatbot uses this information to make multi-session chats feel like working with a CBT therapist. The mental health chatbot can use the logic implemented in the NLP engine 143, the artificial memory engine 163, and the tangential conversation engine 173 to provide CBT therapist-like treatment to patients. For example, when a patient utters “my cat just passed away”, the technology disclosed applies logic that detects the event in the utterance i.e., “death” of the cat. The technology disclosed further includes logic to detect the appropriate sentiment associated with the event. The sentiment can be positive, negative, neutral, etc. In this example, the sentiment is negative as the patient is “sad” due to her loss. An inappropriate prompt to the patient such as “cats are awesome”, or “cats are amazing” in response to the above utterance can destroy the therapeutic relationship between the patient and the chatbot. The chatbot includes the logic to form an appropriate response to the above utterance as explained below.

The technology disclosed includes logic to form an appropriate response that is not only sensitive to the patient's situation in the current utterance but also draws upon the information from the artificial memory to build a therapeutic relationship with the patient. For the above example, the system detects the state (i.e., dead) of the entity (cat) in the utterance. The system also detects a negative sentiment (i.e., loss or grief) associated with the event in the utterance. The system can access the artificial memory database for the patient to query the cat's name e.g., “Mittens”. The mental health chatbot can then respond to the patient by saying something like, “I am sorry to hear about Mittens”. The system also includes logic to present one or more appropriate therapy exercises or coping tools to the patient based on the detected sentiment of the patient towards the entity and the state of the entity. For example, the chatbot can say, “Here are some tools that you may find helpful in dealing with the loss”. The patient may accept to go through a suggested exercise, in which case, the chatbot can bookmark the last completed step in the structured steps of the CBT therapy session and start a tangential conversation. The system can also access the artificial memory database to query positive sentiments associated with the entity (in this case patient's cat) to say something like, “There is a lot you can look back and talk about when you shared ice cream with Mittens”. The technology disclosed can thus provide a high-quality therapy experience to patients by maintaining the patient specific artificial memory graph data structure and making use of remembered content by matching it with appropriate sentiment.

The technology disclosed also includes logic to handle conversational branching or tangential conversations by recognizing when to shift topics from utterances of a patient. The conversational branching enriches the interaction of the patient with the chatbot in a dynamic and flowing conversational pattern meant to emulate a session with a therapist. The natural language processing (NLP) engine 143 includes logic to detect the intent of the patient from their utterances and allows the patient to digress from the linearly structured steps of the CBT session. As the patient engages in multiple CBT sessions with the chatbot they will not always be on task. The chatbot includes logic to entertain tangential conversations for brief or extended periods. The chatbot bookmarks the last completed step in the main flow of the linearly structured steps of the CBT session so that it does not lose track of the goal of the session and state of progress.

The system implements an abstract state machine (ASM) to track the state of progress of a CBT session. ASM is a formalism for modeling sequential state changes between any type of data structure. In our case, these state changes represent transitions between points in a conversational flow. We are using ASM to model not only the linguistic content of the conversation, but also the various updates to memory representations and emotional connotations that this linguistic content gives rise to.

The technology disclosed includes explicit and implicit triggers or conditional events to start a conversational branch. Explicit triggers are straightforward and can include inputs from the patient to start or end a tangential conversation. For example, during a therapy session, the chatbot can ask the patient whether she would like to hear more about a particular therapy technique before moving on. The chatbot can provide “yes” and “no” options to the patient. By selecting the “yes” option, the patient can initiate a tangential conversation. Explicit triggers can also be used to end a tangential conversation and resume the flow of the CBT session from a step following the bookmarked last completed step. Examples of explicit triggers to end a tangential conversation can be a button input. For example, the chatbot can send a prompt to the user “Is this helpful? Should I continue?” Or “Do you want to keep talking about this?”. The patient can respond “no” to end the tangential conversation. The explicit trigger can be a free text input from the patient. For example, the patient can write, “I do not want to talk about this anymore” or “Let's talk about something else.”

The technology disclosed also includes implicit triggers to end a tangential conversation. The implicit triggers can include detecting various factors in patient's utterances to gauge her level of interest in continuing with a particular conversational path. The system can resume the flow of the CBT session when the system detects that the patient's interest is waning. Examples of implicit factors can include sentiment of the utterances, length of patient's utterances as measured in character count, length of the conversation in a branch, etc.

Completing the description of FIG. 1, the components of the system 100, described above, are all coupled in communication with the network(s) 181. The actual communication path can be point-to-point over public and/or private networks. The communications can occur over a variety of networks, e.g., private networks, VPN, MPLS circuit, or Internet, and can use appropriate application programming interfaces (APIs) and data interchange formats, e.g., Representational State Transfer (REST), JavaScript Object Notation (JSON), Extensible Markup Language (XML), Simple Object Access Protocol (SOAP), Java Message Service (JMS), and/or Java Platform Module System. All of the communications can be encrypted. The communication is generally over a network such as the LAN (local area network), WAN (wide area network), telephone network (Public Switched Telephone Network (PSTN), Session Initiation Protocol (SIP), wireless network, point-to-point network, star network, token ring network, hub network, Internet, inclusive of the mobile Internet, via protocols such as EDGE, 3G, 4G LTE, Wi-Fi and WiMAX. The engines or system components of FIG. 1 are implemented by software running on varying types of computing devices. Example devices are a workstation, a server, a computing cluster, a blade server, and a server farm. Additionally, a variety of authorization and authentication techniques, such as username/password, Open Authorization (OAuth), Kerberos, Secured, digital certificates and more, can be used to secure the communications.

### Mental Health Chatbot—System Components

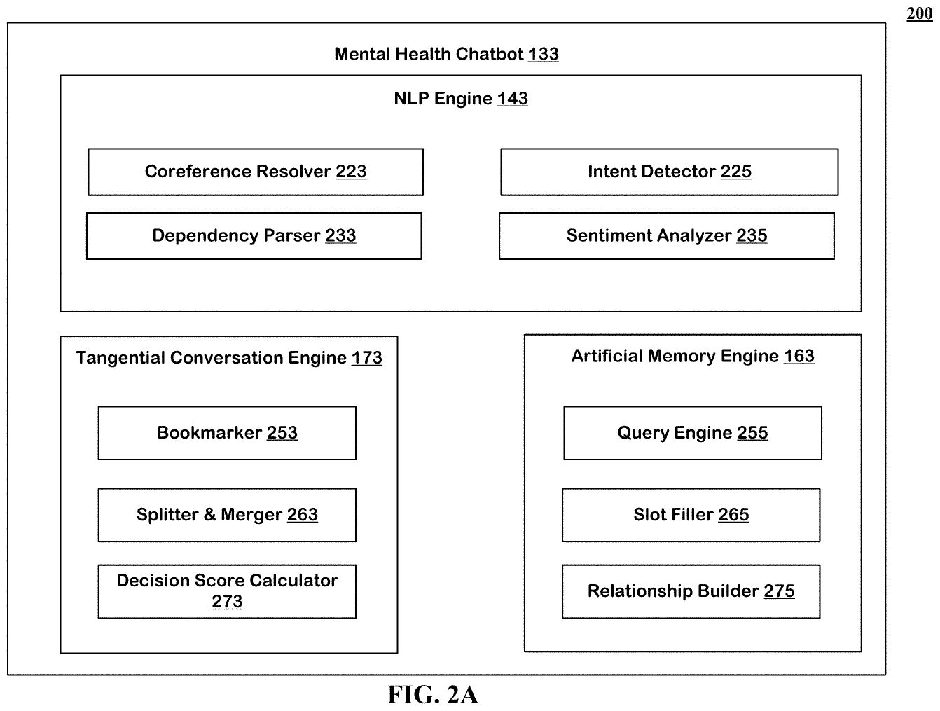

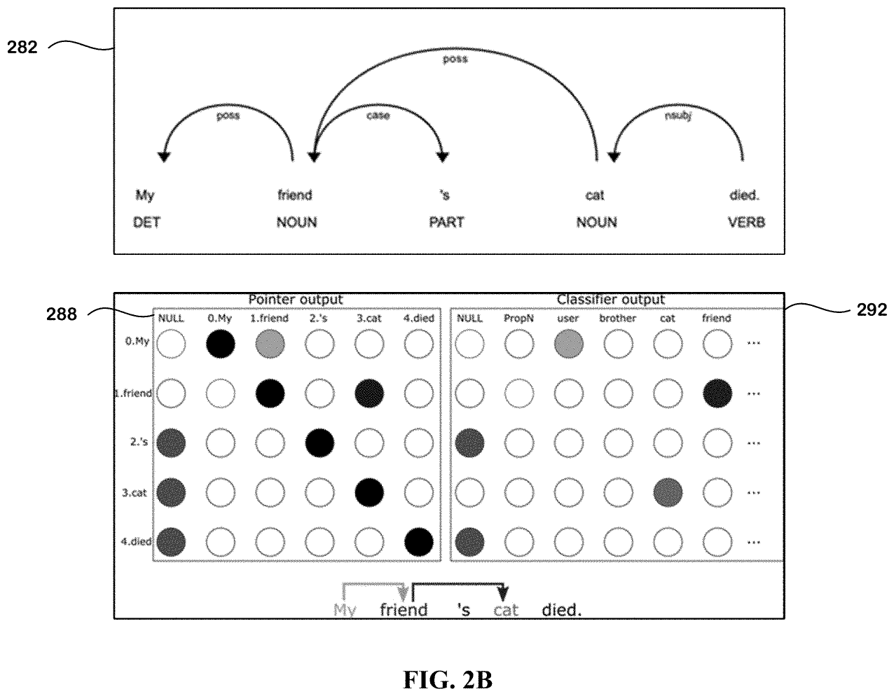

FIG. 2A is a high-level block diagram 200 of components of mental health chatbot 133. The mental health chatbot comprises of three high-level components, the NLP engine 143, the artificial memory engine 163, and the tangential conversation engine 173. The NLP engine further comprises of a coreference resolver 223, an intent detector 225, a dependency parser 233, and a sentiment analyzer 235. The artificial memory engine 163 further comprises of a query engine 255, a slot filler 265, and a relationship builder 275. The tangential conversation engine 173 further comprises of a bookmarker 253, a splitter & merger 263, and a decision score calculator 273. We present details of the components in the following sections.

NLP Engine

The NLP engine 143 includes logic to apply computerized natural language processing techniques to resolve ambiguous relationship references and pronouns used by the patient in an utterance. The NLP engine includes logic to use patterns in dependency parse of resolved utterance along with relationship ontology to extract entities from the artificial memory graph data structure. The NLP engine performs sentiment analysis to detect patient's sentiment towards the entity in the utterance. The NLP engine includes logic to detect a present intent of the patient to diverge from the flow of linearly structured steps of the CBT session and start a tangential conversation. Before applying any of the NLP techniques, the NLP engine performs grammar and spelling correction on patient's current utterance resulting in a corrected utterance. We now describe sub-components of the NLP engine 143 that perform the above listed operations.

Coreference Resolver

The coreference resolver 223 includes logic to resolve pronouns in utterances from patients. For example, consider a patient Bob is talking with a mental health chatbot. During the conversation, Bob says, “My sister has a friend called John”. In response, the chatbot prompts, “Really, tell me more about him”. Bob continues in the next utterance, “She thinks he is so funny”. There are several implicit references in the last utterance from Bob: “she” refers to the same entity as “My sister”: Bob's sister in the previous utterance; “he” refers to the same entity as “a friend called John”: Bob's sister's friend. The process of linking together mentions in a patient's utterance to the entity is called coreference resolution.

The coreference resolver 223 performs coreference resolution on the current utterance and prior corrected utterance (if available) to identify “who” of each pronoun. For example, consider a corrected utterance from a patient “My wife started a new job at Vandelay Industries. Her coworkers all seem very nice.” The pronoun “Her” refers to “My Wife”. The coreference resolver generates a resolved utterance “My wife started a new job at Vandelay Industries. My wife's coworkers all seem very nice.” Therefore, the coreference resolver processes the current utterance and outputs a resolved utterance in which the pronouns are replaced with references. The technology disclosed can apply existing coreference resolution technique such as presented at <medium.com/huggingface/state-of-the-art-neural-coreference-resolution-for-chatbots-3302365dcf30> accessed on Jun. 25, 2020, which is hereby incorporated by reference. The resolved utterance is then processed by dependency parser 233 to extract entities from the artificial memory graph data structure.

Dependency Parser

The dependency parser 233 uses patterns in the dependency parse of the resolved utterance to extract entities in the form of graph traversal. The dependency parser uses the hypernym and hyponyms defined in the relationship ontology as well as their synonyms, to detect one or more relationship edges connecting the entity nodes in the artificial memory graph data structure. The technology disclosed can apply existing dependency parsing techniques to parse the resolved utterances. For example, one such technique is presented by Jurafsky and Martin 2019, “Speech and Language Processing” Chapter 15 available at <web.stanford.edu/˜jurafsky/slp3/15.pdf accessed on Jun. 25, 2020, which is hereby incorporated by reference. Consider an example utterance from a patient, “My friend's cat died”. The dependency parse of the utterance is shown in illustration 282 in FIG. 2B and includes edges connecting the elements or tokens of the utterance [“My, “friend”, “'s”, “cat”, “died”]. The elements of utterance are also labeled with their respective dependency relationship descriptors, “My” is a determiner, “friend” is a noun, “'s” is a particle, “cat” is a noun, and “died” is a verb. The dependency parse as shown in FIG. 2B matches the pattern DET(My)<-[poss]-NOUN(friend)<-[poss]-NOUN(cat).

The technology disclosed can query the artificial memory graph data structure using the relationships identified by dependency parsing of the utterance to extract the entity. The goal is to extract the entity from the artificial memory graph data structure and use the information about the entity to advance the conversation with the patient. The system can use existing graph query languages such as Neo4j's Cypher graph query language (available at neo4j.com/developer/cypher-basics-i/) to query the artificial memory graph data structure. In Cypher query language, the query is formed by using a combination of entities and relationships. Entities are presented in parentheses using variable names or labels. The nodes that are not relevant in the query can be specified in empty parentheses. Relationships are represented by edges between nodes. The language can handle both directed and undirected edges between nodes. For example, to extract the entity in the graph representing patient's friend's cat we can use a query: (user)-[friend]->( )-[cat]->( ) in the pseudo-cypher syntax of Neo4j's query language. (Less desirably, a graph can be implemented using a relational database and traversing the graph using queries and joins.) Note that we are querying the graph starting with the user or the patient entity. The patient is connected to the neighboring entity by a “friend” relationship. We leave the parentheses empty after the “[friend]” relationship as we do not refine the matching entity node with further information. The friend entity is further connected to the target entity by a “[cat]” relationship. Finally, the entity connected to the friend with a “[cat]” relationship is returned when the query is executed. The technology disclosed can elicit further contextual evidence and clarification from the user if the query returns more than one entity to disambiguate and identify a unique entity.

Intent Detector

The intent detector 225 includes logic to classify a patient's conversational intent from a particular utterance. The system can use this classification as one of the inputs to the logic to detect a present intent of the patient to diverge from the flow of linearly structured steps of the CBT session and start a tangential conversation. In natural language processing, an intent categorizes an end-user's intention for one conversation turn. When a chatbot receives an utterance (in verbal or written form), the intent detection engine can match the utterance to one of the many pre-defined intents of the chatbot. The system can use intent detection Application Programming Interfaces (APIs) to detect a change of intent in the utterances of the patient. Examples of such APIs include Google™ DialogFlow.

In general, conversations of users with chatbots can be categorized as structured or unstructured. A structured or linear conversation with a pre-set number of options and outcomes makes a conversation predictable. The conversations with patients in therapy sessions are not structured and therefore, the system includes implicit and explicit triggers to detect when the patient wants to start a tangential conversation. We present details of the implicit and explicit triggers to start and end tangential conversations later in this text. The technology disclosed can apply the implicit and explicit triggers in addition to the intent detection APIs to patient utterances to detect the intent of the patient to shift topics.

Sentiment Analyzer

The sentiment analyzer 235 can include logic to detect a positive, negative or neutral sentiment of the patient in the current utterance. Sentiment analysis is applied in many natural language processing applications. The sentiment analyzer not only detects the sentiment of the patient in the current utterance but also may link that sentiment to the entity mentioned or referenced in the utterance. The entity nodes in the artificial memory graph data structure are slotted to store the sentiment of the patient towards the entity. The relationship edges between the entity nodes can also be slotted to store sentiment of the patient.

Artificial Memory Engine

The artificial memory engine 163 includes logic to remember content received from a patient for future use. The artificial memory engine also includes logic to make use of remembered content during a therapy session from earlier interactions with a patient in a series of sessions. There are three sub-components of the artificial memory engine including a query engine 255, a slot filler 265, and a relationship builder 275. We present details of the three components in the following sections.

Query Engine

The query engine 255 includes logic to build queries for accessing the artificial memory graph database. The system can use existing graph query languages such as Neo4j's Cypher graph query language (available at neo4j.com/developer/cypher-basics-i/) to query the artificial memory graph data structure. The technology disclosed maintains a graph of entities and relationships in an artificial memory graph data structure. The entity nodes represent entities that are related to the patient root node representing the patient by one or more relationship edges that have named roles. The entity nodes and/or the relationship edges are slotted to store attributes such as distinguishing name of the entity, a patient sentiment (e.g., positive, negative, neutral, etc.) towards the entity and a state of the entity (e.g., dead or alive). It is understood that other types of attributes can also be stored in the slots on entity nodes and relationship edges.

The technology disclosed includes entity extraction algorithms that use one or more tokens from utterances of patients for use in queries to extract entities. The chatbot can use the extracted entities and their attributes to advance the conversation with the patient. The technology disclosed can add new entities mentioned in patient's utterances to the artificial memory graph data structure. The technology disclosed extends the existing entity storage and extraction algorithms by creating one or more relationships between the patient root and the entity nodes representing the entities. The system maintains a relationship hierarchy that describes alternative relationship edges used to connect the patient root node to a particular entity node. The relationship hierarchy is structured as hypernym and hyponym relationships. We present two example entity extraction algorithms that can be applied to extract entities from the artificial memory graph.

### Algorithm 1 (Rule-Based Implementation)

Step 1: Perform grammar and spelling correction on patient's current utterance resulting in a corrected utterance

Step 2: Perform coreference resolution on current corrected utterance and prior corrected utterances (in the present therapy session) to identify referent for each pronoun

Step 3: Use patterns in the dependency parse of the resolved current utterance using relationship ontology to extract entity (as described in example of extraction of friend's cat entity in description of dependency parser 233 above).

### Algorithm 2 (BERT Implementation)

The second entity extraction is based on a pre-trained BERT network (Delvin, et al. (2019) BERT: Pre-training of Deep Bidirectional Transformers for Language Understanding, available at arxiv.org/abs/1810.04805, which is hereby incorporated by reference).

The inputs to the model are tokens of a coreference resolved utterance produced by a tokenizer such as BERT tokenizer.

The output from the model is a combination of a Pointer Network (Vinyals, et al. (2017) Pointer Networks, available at arxiv.org/pdf/1506.03134.pdf, which is hereby incorporated by reference) output layer 288 and a classification output layer 292 (see FIG. 2B). We now describe the Pointer Network output 288 and the classifier output 292 as shown in FIG. 2B. The colored circles in outputs 288 and 292 indicate activation of one of a softmax output. Black circles have activation fixed to zero.

The Pointer Network output layer 288 has n (tokens)+1 softmax outputs associated with each token at the input where n is the number of input tokens. The first element of the softmax output is a NULL pointer indicating that the token does not have an outgoing edge to another token while the remaining elements correspond sequentially to an outgoing edge to another token in the utterance. Self-references can be fixed at zero activation in the softmax. In an alternate implementation, the self-references can be interpreted as NULL and the output can be reduced to n softmax outputs.

The classification output layer 292 has n (Types)+2 softmax outputs associated with each token at the input where n (Types) is the number of recognized relationship types such as brother, mother, etc. Each token is classified as either a recognized relationship type, NULL (meaning it is not an entity), or a proper noun class in which the name is extracted from the text of the token.

The output from Pointer Network and classifier in algorithm 2 can be combined into a form that can be used to query the artificial memory graph. An example of this query format is presented above as (user)-[friend]->( )-[cat]->( ) in the pseudo-cypher syntax of Neo4j's query language.

Slot Filler

The slot filler 265 includes logic to fill various information about an entity which is represented by an entity node in the artificial memory graph data structure. The information about the entity is received from patient's utterances. The slots can store data in primitive data types like strings, Booleans, floats, pointer to external resources in global knowledge graph data structure (e.g., WikiData), etc. The edges between entity nodes can have slots that can store data such as sentiment of an entity towards another entity. The system can add more slots to nodes and edges to accommodate new types of information and new data types. We present some example slots and the values that can be stored in respective slots.
type: {‘Person’, ‘Pet’}
givenName: string of a person's given name.
familyName: string of a person's family name.
otherNames: set of strings for other names the person goes by (like nicknames).
firstMentioned: integer timestamp.
lastMentioned: integer timestamp.
alive: {True, False} OR state: {alive, dead}
religion: {‘christianity’, ‘hinduism’, ‘islam’, ‘atheist’, . . . }
education: set of {‘primary’, ‘secondary’, ‘tertiary’, . . . }
livesIn: pointer to WikiData or string name if not in WikiData.
placeOfOrigin: pointer to WikiData or string name if not in WikiData.
workPlace: pointer to WikiData or string name if not in WikiData.
entitySentiment: directed edge between two entities expressing the sentiment of the tail entity towards the head entity. The floating point sentiment score and label from {‘negative’, ‘neutral’, ‘positive’}. The patient's sentiment can also be added to a slot on the relationship edge between the patient root and entity node. A separate “sentiment edge” can also be added between the patient's sentiment towards the entity including the slot for the sentiment of the patient towards the entity.
mentions: sequential list of pointers to messages in an external database where this entity is mentioned and spans of tokens where this entity is detected.

We present two slot filling algorithms that can be used by the slot filler 265 to store data from patient's utterances to slots in entity nodes and/or relationship edges.

### Algorithm 1 (Rule-Based and Classification, Hybrid Implementation)

Rule-based relationship extraction maps entity tokens to other tokens in the utterance that can represent useful information towards filling a slot. We present three high-level steps in this process along with examples. There are two parts of this algorithm. The first part comprises of three steps.

Step 1A: Use output of an entity extraction algorithm (such as presented above) to identify terminal entity tokens of interest in the resolved current utterance. The terminal entity is the target entity in the entity extraction process. For example, the terminal entity is “cat” in “My”->“friend”->“cat” example presented above.

Step 2A: Use patterns in the dependency parse of the resolved utterance to connect entities to tokens that can represent useful information. For example, consider the resolved utterance: [“My”, “friend”, “'s”, “cat”, “died”]. We perform dependency parse of the resolved utterance as presented above and obtain matching pattern such as Noun (cat)<-Verb(died). In this pattern the lemma “die” is a keyword which is recognized as a candidate for the alive (or state) slot in the entity node. The token “died” can thus represent useful information which we can store in the state slot of the cat entity.

Step 3A: The text and lemma of each candidate token is then compared to a set of keywords that might be relevant to a subset of slots in the entity node. These keywords can be used to fill the slot provided the relevance score is sufficiently high or higher than a threshold. The relevance score is obtained from the second part of this algorithm presented below. We illustrate step 3 by continuing the example presented in step 2 above. We identify candidate tokens in [“My”, “friend”, “'s”, “cat”, “died”] example. The lemma “die” of token “died” is a keyword for the alive slot of the cat entity. The relevance score (from part b below) greater than 0.5 for the alive slot indicating that “died” keyword is used literally and therefore, we can fill in the alive slot of the cat entity extracted by the query (user)-[friend]->( )-[cat]->( ) as false, or we can fill in the state slot with the value dead.

Another example, where the relevance score can be less than 0.5 is for an utterance “The audience was alive”. The alive keyword in this utterance is parsed and relevance score is calculated as less than 0.5. This is because the alive keyword is used metaphorically and not literally. Therefore, the alive (or state) slot is not filled.

In the second part of algorithm 1, we use a pre-trained BERT, supra, word embedding model as a classifier to validate whether a token pointed to by the entity extraction algorithm actually represents useful information to fill a slot. We present the three steps of the second part of algorithm below.

Step 1B: The resolved utterance is provided as input to the model.

Step 2B: Each token in the utterance is associated with a binary classification for each slot type that indicates relevance of a token to the slot type. This is important to distinguish between, say, literal and metaphorical uses of keywords.

Step 3B: If the relevance score is higher than a threshold (e.g., 0.5), the slot will be filled based on the keyword and other properties of the parsed utterance. Otherwise, if the relevance score is below the threshold, the slot will remain unchanged.

Training of Model: Model is trained on utterances and binary classes for candidate tokens.

### Algorithm 2 (BERT-Based Regression Implementation)

The input to algorithm 2 is a position of a terminal entity identified using an entity extraction algorithm. The input position can be represented by a transformer model position encoding (Vaswani, et al. (2017) Attention Is All You Need, available at arxiv.org/abs/1706.03762, which is hereby incorporated by reference) and a resolved utterance.

The outputs from the algorithm are classifications or other regressors for each slot that explicitly provides a value to fill in a slot for the entity given as input. For example, the alive or state slot can be a softmax value with classes NULL, “dead”, and “alive”. The other slots such as “workplace” and “placeOfOrigin” slots can be represented by pointers to tokens (Pointer Networks, supra) in the utterance similar to the second entity extraction algorithm above.

The entity extraction algorithms are applied to all utterances from patient. If entities are identified, then slot filling algorithm is applied to the utterance to fill slots for the identified entity.

Relationship Builder

The relationship builder 275 includes logic to add new relationship edges between the patient root and entity nodes and between pairs of entity nodes. It can also add relationship edge between patient root and a new entity node added to the artificial memory graph data structure. Consider the following utterances from the patient during a conversation with the chatbot (also known as Serenity).

Patient: “I am sad because my friend backstabbed me at work”.

The system applies entity extraction algorithm to form a query (user)-[friend]->( ) which in this instance results in multiple entity nodes. The system can apply additional logic to filter the extracted entities. For example, the system can filter out all entities that are not patient's coworkers. This results in two entity nodes with distinguishing names (or labels) “Mike” and “David”. As there are two nodes, the system needs to disambiguate before filling in the sentiment slot of an entity. Thus, the chatbot sends the following prompt to the patient that includes the distinguishing names of the extracted entities.

Serenity: “I am sorry to hear about that”.
Serenity: “Would this happen to be about Mike or David?”
Patient: “No, this is about Ted.”

From the above utterance, we can see that patient has not selected any one of the extracted entities from the artificial memory and instead, the patient has provided the name of a new entity. The system applies entity extraction algorithm to the current utterance. The queries (user)-[friend]->( ) and (user)-[coworker]->( ) do not return any entity node with distinguishing name “Ted”. Thus, the system creates a new node with “givenName” or “distinguishing name” slot filled in with “Ted”. The system then creates a new relationship edges between the patient root node and the entity node with relationship roles as “friend” and “coworker” respectively. The relationship builder uses the following queries to create these relationship edges (user)-[friend]->(Ted) and (user)-[coworker]->(Ted). Finally, the slot filler fills in the sentiment slots of the new entity with a negative sentiment from the patient towards the newly created entity node. The system can also fill in sentiment to a slot in a relationship edge between the patient root and patient node. In another implementation, the system can add a sentiment edge between the patient root and entity node which include a slot to store the patient's sentiment toward the entity.

Tangential Conversations Engine

The tangential conversations engine 173 includes logic to detect when to diverge to a tangential conversation from linearly structured steps of a CBT session. It also includes logic to detect when to merge back to flow of linearly structured steps of the session from the tangential conversation in the therapy session. The questions/and or prompts provided to the patient may be pre-determined but the patient's response is unknown and therefore, the technology disclosed includes logic to detect intent in patient's utterances in addition to the entity extraction and sentiment analysis as described earlier. During a therapy session of a linearly structured CBT session, the patient can provide any number of responses to a prompt such as “What thoughts do you have?”. The patient can respond to this prompt as “I am thinking about walking my dog in Richmond Hill later” or the patient can provide any number of responses ranging from totally irrelevant information to expressions of self-harm. During the conversations in a therapy session, the questions from the chatbot can be open ended resulting in responses from the patient that are unknown and unpredictable.

The technology disclosed includes logic to enable the mental health chatbot to progress through the CBT module and still entertain tangential conversations for brief or extended periods. The tangential conversations engine recognizes from patient utterances a patient's intent to shift topics or digress. The tangential conversations engine can include three sub-components, a bookmarker 253, a splitter and merger 263 and a decision score calculator 273.

Bookmarker

The bookmarker 253 includes logic to not lose track of the CBT goal and progress made in the current therapy session by bookmarking the last completed state before starting a tangential conversation. The bookmarking provides ability to return to the predefined sequence of the CBT therapy session when the patient digresses and a tangential conversation is started. The bookmarker uses the abstract state machine construct to make note of the last completed step in the series of steps in the therapy session before the user goes off the predefined path to a tangential conversation branch. When the tangential conversation is concluded, the system knows the last state to return to in the CBT therapy session via the bookmark. Tangential conversations can leverage bookmarking to return back to the last completed step of the CBT therapy session and then continue progress to next steps in the linearly structured steps of the CBT therapy session.

Splitter and Merger

The splitter and merger 263 includes logic to diverge to a tangential conversation from linearly structured steps of the CBT session and to merge back to flow of linearly structured steps of the session from the tangential conversation. The path of conversation between a chatbot and a patient can be more defined and definite in a system that limits variability in the input a user can provide. In such systems the path of conversation will follow seamlessly from the first step to the last step without any divergence. However, humans do not communicate this way, especially in a therapy session. Splitter and merger 263 includes logic to process the triggers or conditioning events in utterances of a patient to start and end tangential conversations. The technology disclosed can process types of triggers to start the tangential conversations, explicit triggers and implicit triggers.

Explicit triggers are straightforward. They are present in the utterances when a patient directly expresses a desire to begin or end a tangential conversation or a tangential path. Suppose the chatbot asks a patient whether she would like to hear more about a particular therapy technique before moving on. The patient is given a choice of “yes” and “no” buttons for input. By choosing “yes”, the patient can start a tangential conversation path that can eventually loop back to the bookmarked point. Explicit triggers can also be used to end a tangential conversation path. For example, an explicit trigger to end a conversation path can be a button input such as the chatbot can ask the user in a tangential path, “Is this helpful? Should I continue?” or something like “Do you want me to keep talking about this?” The patient can say “no” in response to the above prompt to end the tangential conversation. The patient can also say something like “I do not want to talk about this anymore” or “Let's talk about something else” to end a tangential conversation. We now present some examples of tangential conversation triggered by explicit triggers. The chatbot is referred to as Serenity. The conversations shown are small portions of conversations in therapy sessions and are presented for illustration purposes.

Example 1: START tangential path, triggered by CHOICE BUTTON input
[ . . . ]
Serenity: “Would you like to hear more about this therapy technique before moving on?”
Patient: [button input] Yes
[Tangential path begins ]
Serenity: “OK, here's some more information . . . ”
Example 2: START tangential path, triggered by FREE TEXT input
[ . . . in the middle of a previous unrelated topic . . . ]
Patient: “Hey, could we talk about my upcoming test?”
[Tangential path begins]
Serenity: “Sure, what are you feeling about that?”
Example 3: END tangential path, triggered by CHOICE BUTTON input
[ . . . within a tangential path . . . ]
Serenity: “Do you want to tell me more about this, or go back to talking about your day?”
Patient: [button input] “Go back”
[Tangential path ends, return to bookmarked point in conversation]
Example 4: END tangential path, triggered by FREE TEXT input
[ . . . within a tangential path . . . ]
Patient: “Can we talk about something else? This is boring.”
Serenity: “OK, let's go back to talking about [bookmarked topic]”
[Tangential path ends, return to bookmarked point in conversation]

The technology disclosed also includes logic to process implicit triggers to end a tangential conversation. The system includes logic to use a variety of factors to detect patient's waning interest in the tangential conversation. When the system detects that the user's interest level in continuing a tangential conversation is waning, the system can end the tangential conversation and resume the conversation in the main path. The system can also send a prompt asking the patient if she wants to continue with the conversation or go back to the original conversation from the bookmarked position. In the following section, we present examples of implicit triggers to end a tangential conversation.

Decision Score Calculator

The decision score calculator 273 can include logic to calculate a score to determine patient's interest in the tangential conversation. The system can use various factors to gauge patient's interest in continuing with a conversational path. When the interest score or interest level falls below a threshold, the system can send a prompt to the patient suggesting to end the tangential conversation and resume the bookmarked conversation. Example of factors that can be included in calculation of decision score or patient's interest level include the following:

a) Sentiment of the conversation

b) Mood of the conversation.

c) Topic of the conversation

d) People and entities referenced in the conversation.

e) Length of the conversation

f) Length of the user's responses, as measured in word or character count.

The decision score calculator can use a linear or non-linear combination of two or more factors listed above to calculate a score indicating patient's interest in the conversation. The score can provide an estimate of the degree to which the conversation is veering off topic or the patient's interest is waning. If the score exceeds a threshold, the system can send a prompt to the user asking if she wants to continue the conversation or resume the CBT therapy session from the bookmarked position. The system can also use a trained machine learning model that can predict when the user wants to end the tangential conversation. The model can be trained using labeled training data that includes implicit and/or explicit triggers from patients. We present example of using implicit triggers to end a tangential conversation.

Example 5: END path based on implicit triggers
[ . . . within a tangential path . . . ]
Serenity: “What are some challenges you're facing at work?”
Patient: “just stuff”
Serenity: “Hm, what are some positive aspects of your work?”
Patient: “meh”
Serenity: “How do you feel about your coworkers?”
Patient: “ok”
Serenity: “Are there any particular goals you have at work?”
Patient: “n”

We can see in the above example that length of patient's responses is short and is decreasing. The conversation has continued for several iterations without an intense response from the patient. The sentiment scores for the patient's response are neutral. The patient is not brining up any other entity or herself in her utterances. Decision score calculator or implicit triggers model predicts that Serenity chatbot should send a prompt to end the conversation or suggest a new topic for conversation such as presented below:

Serenity: “Hey, do you want to talk about something else?”
Patient: “Yes! Let's talk about my upcoming vacation!”
[Tangential path ends]

### Example CBT Scripts

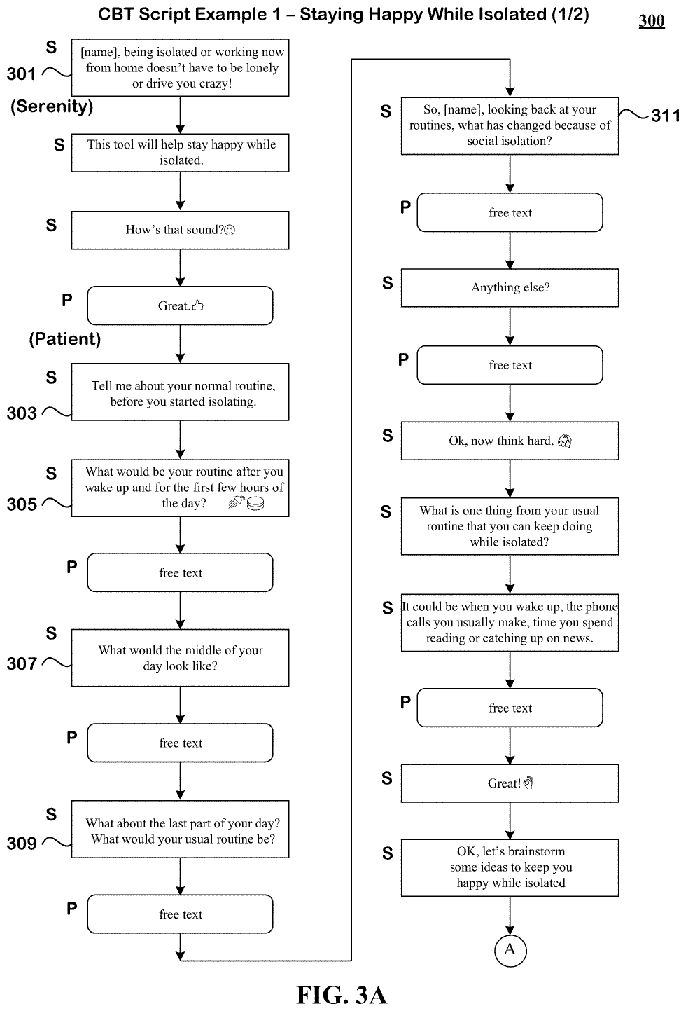

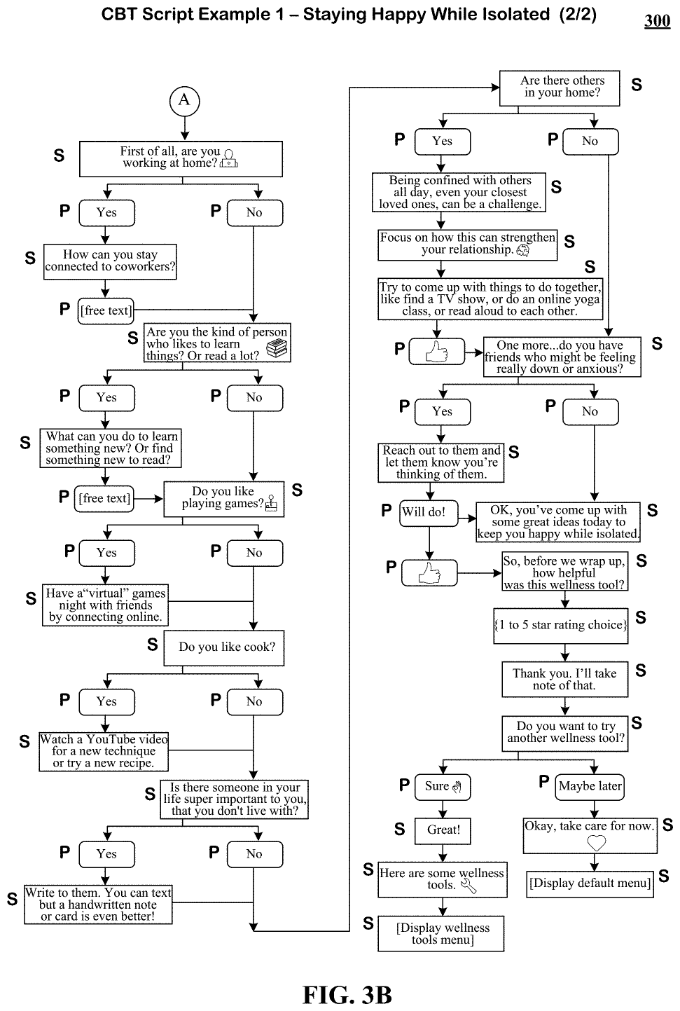

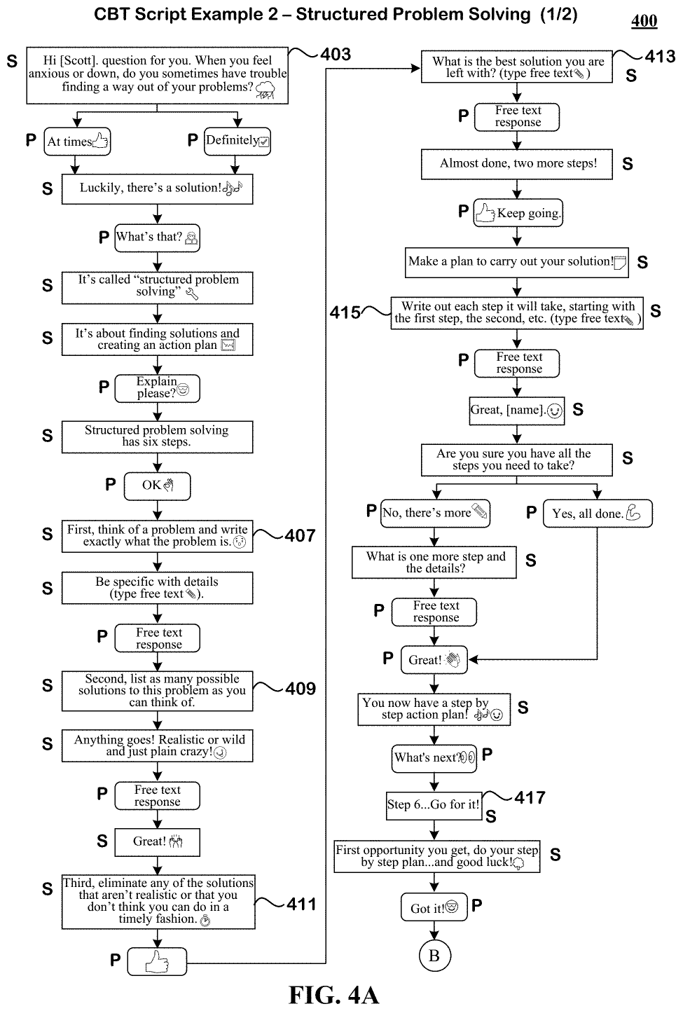

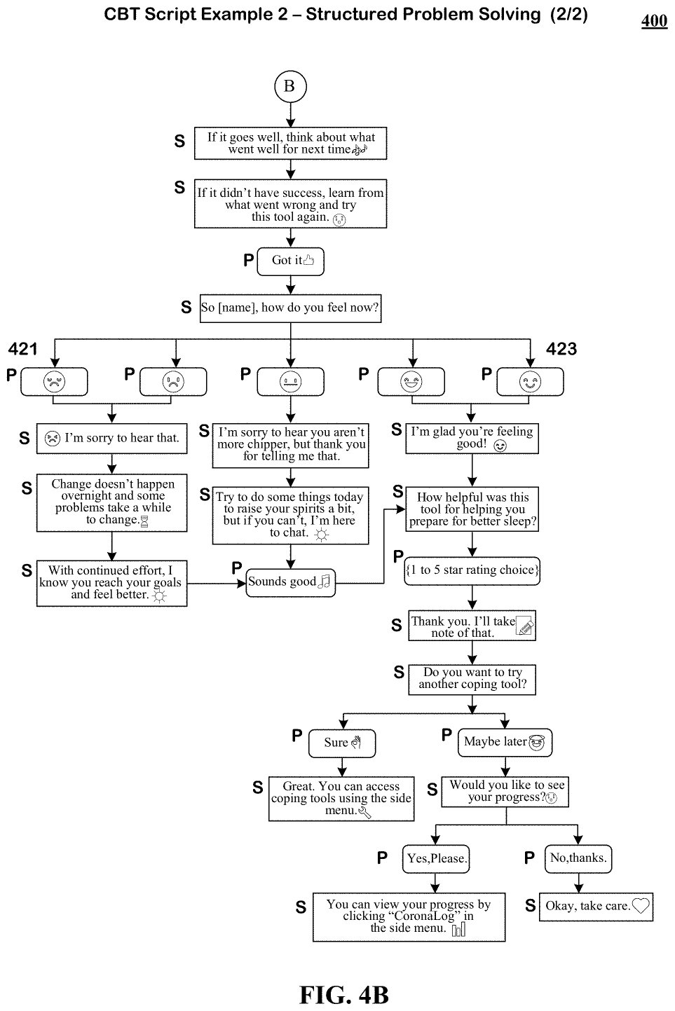

We present two example CBT scripts in FIGS. 3A to 4B. Note that the boxes in the flowchart shown in FIGS. 3A to 4B that contain messages from Serenity are labeled with an “S” to indicate that the prompt is from the chatbot. Patient's utterances are presented in boxes with round edges and are labeled with “P”.

### CBT Example 1: Staying Happy while Isolated

In the first example CBT script presented in FIGS. 3A and 3B, the Serenity chatbot converses with a patient who is in isolation in her home. The example script 300 is presented in two parts, the first part starts at a step 301 in FIG. 3A. The second part is presented in FIG. 3B. The example script can be part of a CBT therapy session and a tangential conversation due to a trigger in utterances of the patient. The patient may have said that she is feeling lonely while isolated at home. The chatbot helps the patient identify activities that she can still do while being isolated. In the first part of the conversation, the chatbot elicits patient's activities (303) during different parts of the day such as at the start of the day (305), middle of the day (307) and the last part of the day (309). The chatbot then talks to the patient to elicit how her routine has changed due to isolation at home (311).

In the second part of the first example CBT script, presented in FIG. 3B, the chatbot converses with the patient to identify activities she can still do while being in isolation that can help overcome the loneliness. Note, the chatbot attempts to elicit patient's interests before suggesting activities. For example, if the patient likes to learn new things or read books, the chatbot can suggest books to read. If the patient likes to play online games, the chatbot can suggest virtual games that can connect patient with friends. The chatbot also helps patient to identify different ways in which she can connect with her friends and family. At the end, the chatbot prompts the patient if she would like to try another wellness tool. If the patient accepts the invitation, the chatbot can present the wellness tool to the patient, otherwise, the chatbot can end the conversation. At this point, the system can resume the main therapy session from the bookmarked position.

### CBT Example 2: Structured Problem Solving

The second CBT script presents an example in which the patient learns about structured problem solving technique. Beck et al. in their book Cognitive Behavior Therapy Basics and Beyond, 2^{nd} edition, present ten principles of CBT. The principle number six listed in their book states, “Cognitive behavior therapy is educative, aims to teach the patient to be her own therapist, and emphasizes relapse prevention”. The therapist sets goals, identifies and evaluates thoughts and beliefs of the patient, and attempts to bring about behavioral change. In addition, the therapist also teaches the patient how to do so on her own so that she can benefit from her new understanding in the ensuing weeks and after treatment ends. The second example of CBT script in FIGS. 4A and 4B teaches structured problem solving technique to a patient. The chatbot asks the patient “when you feel anxious or down, do you sometimes have trouble finding a way out for your problems?” (403).

The chatbot educates the patient about a structured problem solving skill that has six steps. The teaching of the problem solving technique involves working with the patient to go through an example. In the first step, the chatbot asks the patient to think of a problem and write it down (407). The second step is to list as many possible solutions as the patient can think of, to solve the problem (409). The third step is to eliminate the solutions that are not realistic or the solutions that the patient cannot complete in a timely manner (411). In the fourth step, the chatbot asks the patient to write down the best solution left after elimination in the previous step (413). The fifth step is to make a step by step plan to carry out the solution (415). Finally, step 6 is to apply the step by step solution to solve the problem (417).

The second part of the example CBT script for structured problem solving is presented in FIG. 4B. In this part, the chatbot converses with the patient to see how the structured problem solving technique can be applied by the patient herself when she encounters a problem. The last part of the CBT therapy session elicits patient's feedback about the therapy session. Based upon patient's responses ranging from poor (sad face, 421) to excellent (happy face, 423), the chatbot advances the conversation to alleviate her concerns if she is still not feeling happy. Finally, the chatbot asks the patient if she wants to try another coping tool. If the user accepts the invitation, the chatbot guides the patient to access the user interface menu to browse and select a coping tool. If the patient does not want to try another coping tool, the therapy session ends.

### Artificial Memory—Relationship Graph

The Artificial Memory data structure is modeled as a graph that can include a personal relationship graph that is populated with private, patient-specific information about personal relationships (e.g. sister, father, friend, etc.) and a universal Global Knowledge graph that expresses the user's connection to global information (e.g. locations, medical conditions, etc.). In this section we explain the relationship graph data structure with an example.

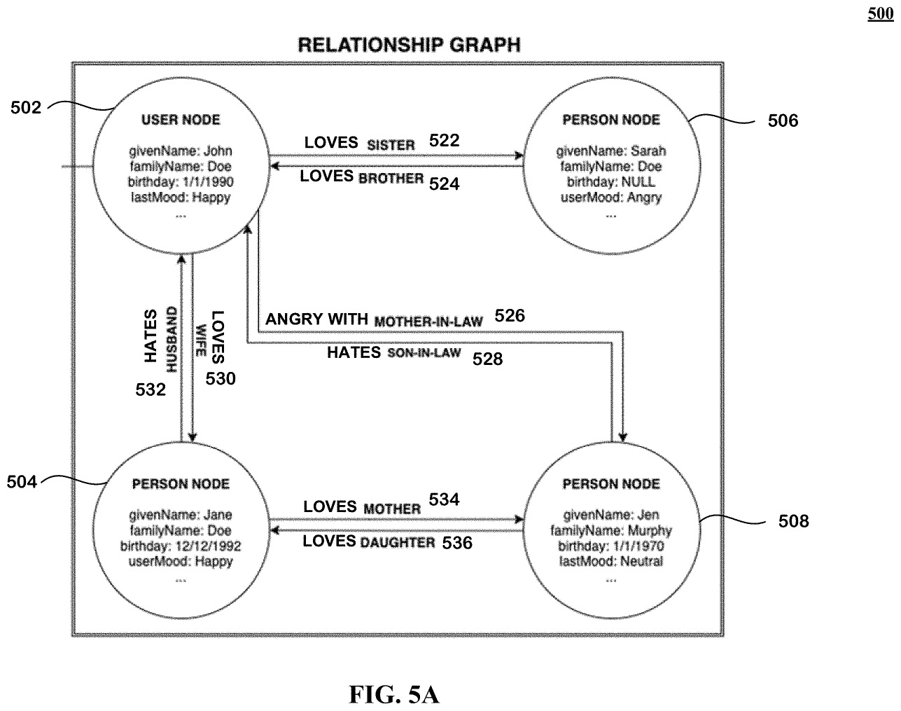

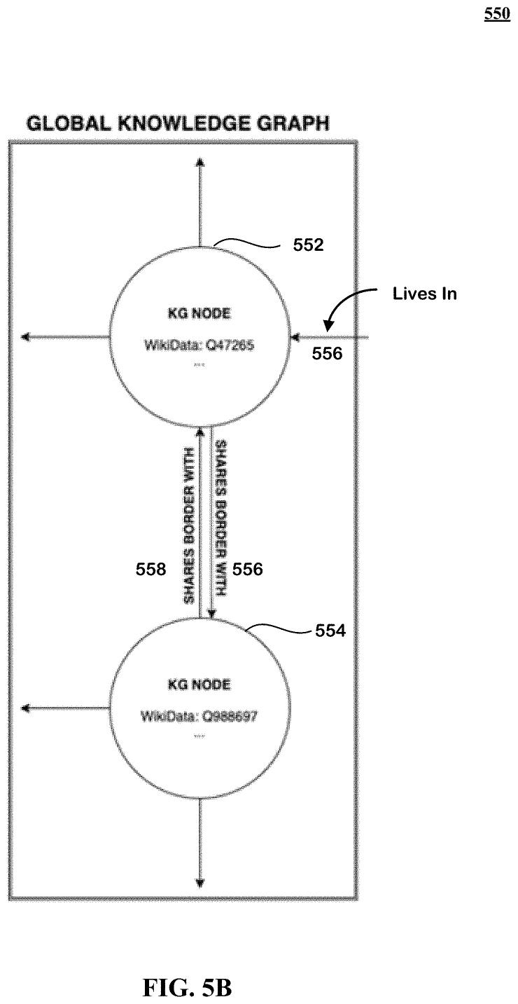

FIG. 5A presents an example artificial memory graph data structure 500 for storing entities and relationships. The data structure is also known as a relationship graph. The system can store entities and relationships of entities to the patient in the relationship graph using the information in utterances of a patient. The remembered content can then be used in therapy sessions. The system continuously updates the entities and relationships as new information is provided by the patient.

The entity nodes are slotted to store entity related attributes. For example, the nodes shown in example have slots for a givenName, familyName, birthday, lastMood, etc. These attributes are shown as an example, the system can include more attributes in the entity node such as an entity state or alive status, workplace, etc.

The relationship graph consists of entity nodes representing entities that are related to the patient root node representing the patient by one or more relationship edges that have named roles. For example, in FIG. 5A, the patient root node 502 (labeled as user node) is connected to three entity nodes, labeled as a person node 504, a person node 506, and a person node 508. The three entity nodes are connected to the patient root node by relationship edges that have named roles. For example, the entity node 504 is connected to the patient by two directional relationship edges 530 and 532. The relationship edge 530 is directed from the patient root node 502 to the entity node 504 and has a relationship named role “wife” indicating that the person node 504 represents an entity that is patient's wife. The relationship edge 532 shows that the patient 502 is husband of the entity 504. The relationship edges can also be slotted to store attributes such as sentiment of the patient towards the entity or the sentiment of the entity towards the patient. Note that these sentiments are elicited from the utterances of the patient and can be updated over time.

The other two entity nodes 506 and 508 are also connected to the patient root node 502 by relationship edges. The person node 506 represents an entity that is “sister” of the patient (relationship edge 522). The relationship edge 524 shows that the patient is a brother of the entity represented by the entity node 506. The relationship edges 522 and 524 also include sentiments of the patient towards the entity (patient loves his sister) and the sentiment of the entity towards the patient (patient's sister hates him). The entity node 508 represents an entity that is “mother in law” of the patient (relationship edge 522). The relationship edge 528 shows that the patient is a son-in-law of the entity represented by the entity node 508. The relationship edges 526 and 528 also include sentiments of the patient towards the entity and the sentiment of the entity towards the patient as shown on relationship edges 526 and 528, respectively.

### Artificial Memory—Global Knowledge Graph

FIG. 5B presents an example of a global knowledge graph (also referred to as universal global knowledge graph) data structure 550. The nodes in the global knowledge graph data structure represent patient's connection to global information. For example, the nodes in the global knowledge graph can represent location, medical conditions, or other global and public information that is related to the patient. The global knowledge graph is not patient specific and therefore, more than one patient root nodes can be connected to nodes in the global knowledge graph. The example global knowledge graph data structure shows nodes that reference WikiData entries. During one of the utterances, the patient may have said that he lives in Palo Alto, Calif. The system then connects the patient root node to the knowledge graph (KG) node 552 that has a reference to WikiData entry for Palo Alto.

The nodes in the global knowledge graph can be slotted to store information. The patient root node or other entity nodes can be connected to one or more nodes in the global knowledge graph data structure using relationship edges. The relationship edges can represent the relationship of the patient or the entity to the node in the global knowledge graph. For example, in the example 550, the patient root node (not shown in FIG. 5B) can be connected to the node 552 by a relationship edge 556 labeled “lives in” indicating the patient lives in Palo Alto, Calif. The relationship edges can also be slotted to store sentiment of the patient towards the place or entity represented by the node in the global knowledge graph.

### Hypernyms and Hyponyms

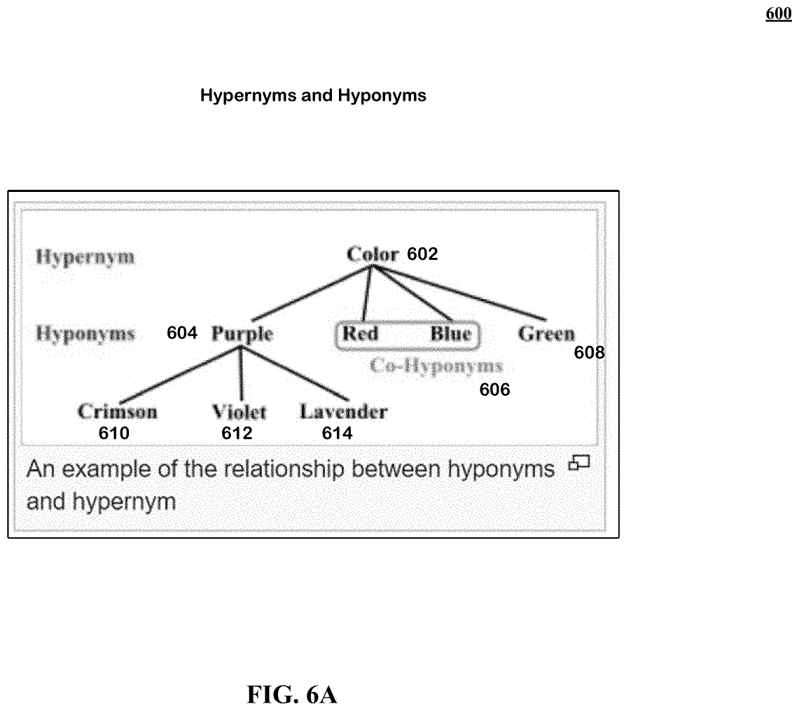

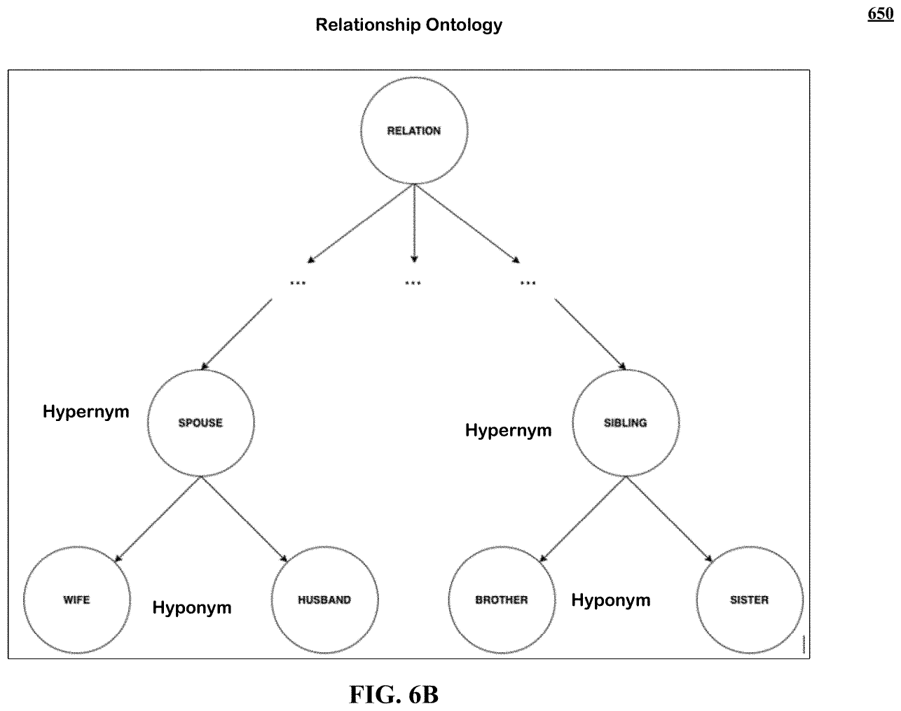

The technology disclosed maintains a relationship hierarchy that describes alternative relationship edges used to connect the patient root node to a particular entity node. The relationship hierarchy is structured as hypernym and hyponym relationships. In linguistics, a hyponym is a word or phrase whose semantic field is included within that of another word, its hypernym. FIG. 6A presents an example hierarchy of hypernym and hyponyms. At the root, “color” is a hypernym with hyponyms purple 604, red, blue (collectively 606), and green 608 are shown. The purple color can be considered as hypernym for three hyponyms crimson 610, violet 612, and lavender 614. From this example, we can see that hypernym and hyponyms is a transitive relationship. For example, violet 612 is a hyponym of purple 604 and purple is a hyponym of color 602. Therefore, violet 612 is a hyponym of color 604. A word can be both a hypernym and a hyponym. For example, purple is a hyponym of color but itself is a hypernym of the broad spectrum of shades of purple between the range of crimson and lavender. We have structured the relationships between the patient root and entities in the relationship graph as a hierarchy of hypernyms and hyponyms as presented in the following section.

### Relationship Ontology

FIG. 6B presents an example relationship ontology or taxonomy in which the root (top-level node) can be denoted by a generic label or tag such as “relation”. The lower levels in the hierarchy can be categorized into less ambiguous terms. For example, siblings can be a parent in the hierarchy or hypernym for sister and brother. Similarly, spouse can be hypernym for husband and wife. The technology disclosed can use the relationship ontology to assign labels to relationship edges. The system can add additional edges when more details of the relationship between the entity and the patient are voluntarily provided by the patient. In one instance the system can add multiple relationship labels to the same relationship edge instead of adding multiple edges with individual relationship labels. Consider an example, in which the patient says “my spouse” in an utterance. The system may already have created a relationship with label “wife” between the patient and his wife. The system can then use the relationship ontology to resolve that spouse is a hypernym of wife and thus resolve the ambiguity. The system can resolve the spouse to wife relationship and use that to traverse the graph to the target entity node.

### Artificial Memory Examples

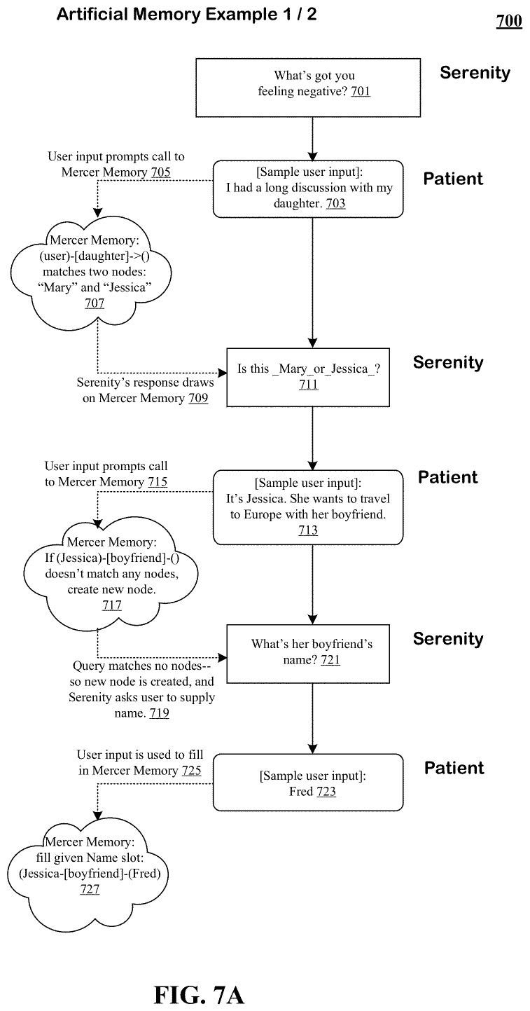

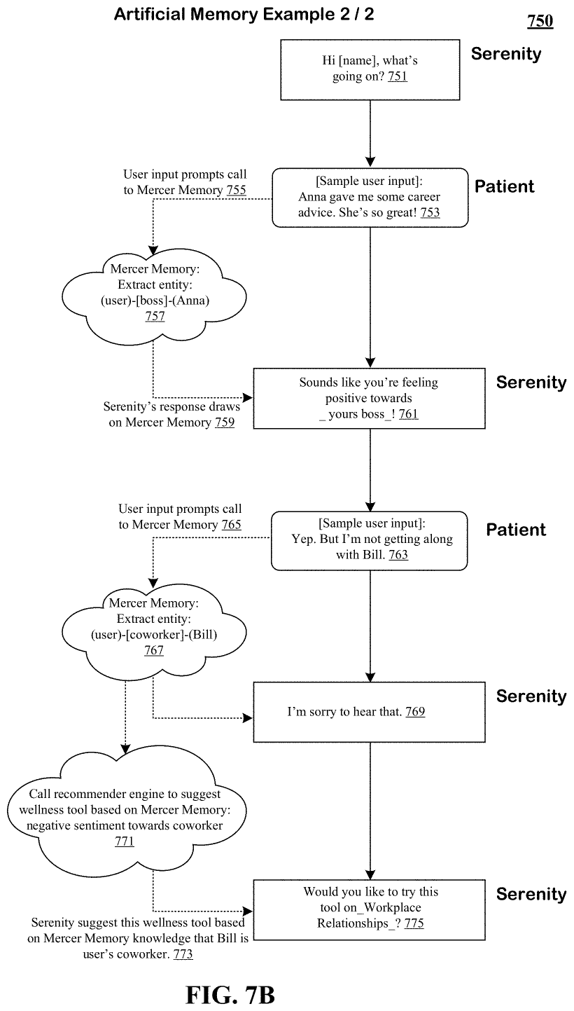

FIGS. 7A and 7B present examples of conversations of Serenity mental health chatbot with patients in which the system queries artificial memory graph data structure using information provided by the patients. In the first example presented in FIG. 7A, the query returns multiple entity nodes with same relationship (daughter) to the patient. The system uses the returned results and prompts patient to disambiguate. In the second example, the system queries artificial memory graph using a distinguishing name of the entity in the patient's utterance to extract the role from relationship edge between the patient root node and the entity in the utterance. The system can then use the extracted relationship to advance the conversation.

### Example 1: Resolve Multiple Entities and Identify New Entity

FIG. 7A presents an example conversation in a CBT therapy session starting with step 701 in which the Serenity chatbot prompts “What's got you feeling negative?” to the patient. Note that in prior utterances to step 701, the patient may have said that she is feeling sad which caused the chatbot to send the prompt 701 to the patient. The patient responds “I had a long discussion with my daughter.” (703). The technology disclosed can detect an event (discussion) involving the entity which is extracted from the artificial memory. The user's utterance initiates the query to artificial memory (or Mercer memory) 705. The system forms a query (user)-[daughter]->( ) to extract the entity related to the patient with a relationship role “daughter”. The query returns two results Mary and Jessica (707). As the query returns a plurality of entity nodes connected to the patient root node by the selected relationship edges, the chatbots response (709) includes the distinguishing names of returned entities, “Is this Mary or Jessica?” (711). The patient clarifies in the next utterance that she is referring to Jessica (713).

The patient provides additional information about a new entity referred to as “boyfriend of Jessica” in her utterance (713). The system builds a new query to extract information about Jessica's boyfriend from artificial memory graph, (Jessica)-[boyfriend]-( ), however, the query does not return any results as this is the first time patient has brought up this entity in her utterance (717). As the query does not match any entity nodes in the artificial memory graph, the chatbot asks the patient to provide name of the new entity (719). The chatbot can create a new entity node in the artificial memory graph data structure and connect it with patient's daughter Jessica's entity node by a relationship edge with a relationship role “boyfriend”. The chatbot sends a prompt to patient to get more details about this new entity, “What's her boyfriend's name?” (721). The patient provides the name of the new entity in her utterance “Fred” (723). The patient's input is used to fill the artificial memory with the new entity's information (725). The system forms a query Jessica-[boyfriend]-(Fred) which will fill the distinguishing name for the new entity node (727).

### Example 2: Extracting Relationship Role Using Entity Name

FIG. 7B presents an example in which the patient provides an entity's distinguishing name to the chatbot in her utterance. The conversation starts with a prompt “Hi [name], what's going on? (751). The patient responds, “Anna gave me some career advice. She is so great!” (753). The system uses the patient's utterance to access the artificial memory to extract more details about the entity mentioned in patient's utterance (755). The query (user)-[boss]-(Anna) returns the relationship “boss” for the entity Anna mentioned by the patient (757). The chatbot uses the query response (759) to send a prompt to the patient “Sound like you're feeling positive towards your boss!”.

The patient then continues the conversation by uttering, “Yep. But I'm not getting along with Bill” (763). The system uses the input from the patient (765) to query the artificial memory. The query (user)-[coworker]-(Bill) returns the relationship of the entity “Bill” to the patient as her coworker (767). The system prompts to the patient “I'm sorry to hear that” (769). The system uses a recommender engine to suggest a coping or wellness tool to the patient using the relationship of the entity to the patient extracted from the artificial memory (771). The tool is based on the fact that Bill is patient's coworker (773). The chatbot sends a prompt to the patient, “Would you like to try this tool on Workplace Relationships?” If the patient accepts the invitation, the chatbot continues to work with the patient through the coping or wellness tool therapy steps.

We now present further examples of using artificial memory during conversations of chatbot with patients.

### Example 3—Remembering Content in Artificial Memory

Patient: “My cat passed away.”
Entity Extraction: (user)-[cat]->( ) matches a unique node. Extract the name “Mittens” from the entity's “givenName” slot.
Slot Filling: The system recognizes the relationship “cat”->“died” indicates that the slot alive is filled in as False on the node extracted from (user)-[cat]->( ). Sentiment analysis is performed and an edge is created/updated between the user and cat node with negative sentiment (sadness).

Serenity: “I am sorry to hear about Mittens. Here are some tools you may find helpful in dealing with loss.”

### Example 4—Remembering Content in Artificial Memory

Patient: “My wife got a promotion at her job at Vandelay Industries.”
Entity Extraction: (user)-[wife]->( ) extracts a unique node. Extract the name “Sarah” from the entity's “givenName” slot.
Slot filling: recognize the relationship “wife”->“Vandelay Industries” and fill in “workplace” slot on (user)-[wife]->( ) node with “Vandelay Industries” or an address to a WikiData object on the industry (global knowledge graph).

Serenity: “How is Sarah liking her job at Vandelay Industries?”

Patient: “Her coworkers all seem very nice.”
Extracted entities: (user)-[wife]->( )-[coworkers]->( ) returns a set of nodes corresponding to Sarah's coworkers.
Slot Filling: sentiment analysis is performed and directed edges are created/updated from the user's wife to her coworkers expressing a positive sentiment.

Serenity: “Glad to hear Sarah is having a positive experience with Vandelay Industries.”

### Example 5—Making Use of Remembered Content & Resolving Multiple Entities with Same Relationship

Patient: “I am sad because my friend backstabbed me at work.”
Entity Extracted: (user) and (user)-[friend]->( ) the latter of which is ambiguous as it connects to multiple nodes. Number of nodes can also be reduced by context, noting that the friend is also likely a coworker. This results in two nodes: “Mike” and “David”.
Slot Filling: sentiment analysis is negative towards (user)-[friend]->( ) but disambiguation is necessary.

Serenity: “I am sorry to hear about that.”

Serenity: “Would this happen to be about Mike or David?”

Patient: “It is Mike this time.”
Entity Extracted: Since we are clarifying, we select “Mike” from the set (user)-[friend]->( ) and (user)-[coworker]->( )
Slot Filling: create/update the sentiment edge from user to “Mike”.

### Example 6—Making Use of Remembered Content & Adding New Entity

Patient: “I am sad because my friend backstabbed me at work.”
Entity Extracted: (user) and (user)-[friend]->( ) the latter of which is ambiguous as it connects to multiple nodes. Number of nodes can also be reduced by context, noting that the friend is also likely a coworker. This results in two nodes: “Mike” and “David”.
Slot Filling: sentiment analysis is negative towards (user)-[friend]->( ) but disambiguation is necessary.

Serenity: “I am sorry to hear about that.”

Serenity: “Would this happen to be about Mike or David?”

Patient: “No, this is about Ted.”
Entity Extracted: Since there is no user named “Ted” in the (user)-[friend]->( ) and (user)-[coworker]->( ) sets, create a new node with “givenName” slot filled in with “Ted” and create new edges (user)-[friend]->(Ted) and (user)-[coworker]->(Ted).
Slot Filling: create/update the sentiment edge from user to “Ted”.

### Example 7—Check-In by Serenity Chatbot by Making Use of Remembered Content

Serenity: “Hello again! Have things been going better with your coworker and friend Ted since we last talked?”

### Example 8—Wellness Tool Recommendation by Making Use of Remembered Content

Patient: “I am having difficulty dealing with my family.”
Entity Extracted: (user)-[family]->( ) which will probably link to many nodes.
Slot Filling: Create/update sentiment edges to family members.
Recommendation Engine: Notice from the relationship graph that the user recently had a child.

Serenity: “Here are some tools you might find helpful.”
[Postpartum depression]
[Family counseling]

### Example 9—Wellness Tool Recommendation Based on Extracted Relationship

Patient: “Susan and I had another fight last night.”
Entity Extracted: (user)-[wife]->(Susan).
Slot Filling: Update sentiment edge from user to “Susan”.
Recommendation Engine: notice from the relationship graph that Susan is user's wife, recommend appropriate wellness tools accordingly.

Serenity: “Sorry to hear that! Here are some tools you might find helpful:”
[Marital trouble]
[Healthy disagreements with your partner]

### Example 10—Wellness Tool Recommendation Based on Extracted Relationship

Patient: “Mary and I got into a huge argument.”
Entity Extracted: (user)-[boss]->(Mary).
Slot Filling: update sentiment edge from user to “Mary”.
Recommendation Engine: notice from the relationship graph that Mary is user's boss, recommend appropriate wellness tools accordingly.

Serenity: “Sounds like you're having workplace trouble. Here are some tools you might find helpful:”
[Navigating workplace relationships]
[Dealing with office politics]

### Tangential Conversations

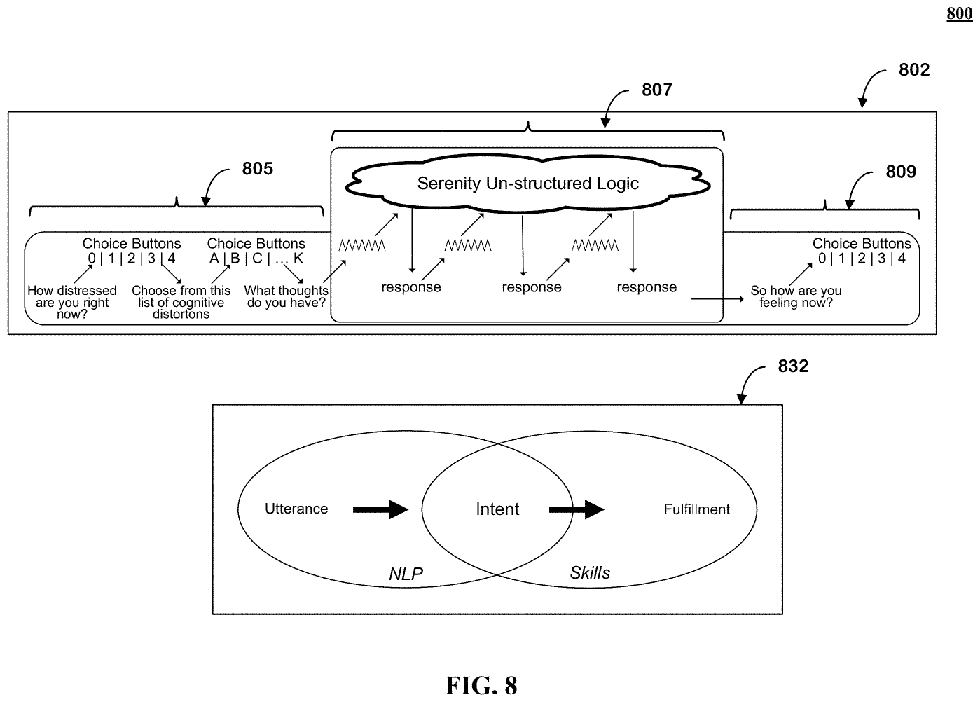

An unstructured, or nonlinear, conversation has an infinite number of possible options and outcomes that make a conversation unpredictable in nature. The questions and/or prompts provided by Serenity chatbot might be pre-determined, but a patient's response is unknown, and thus Serenity's counter-response is also unknown. FIG. 8 presents a conversation consisting of unstructured and structured conversations. The parts of the conversations labeled 805 and 809 are structured. The middle part of the conversation labeled 807 is unstructured. As we can see in a structured conversation, the patient has pre-defined response options to choose from when responding to questions and prompts from the chatbot. On the other hand, when conversations are more unstructured, and open ended in nature, the chatbot is exposed to a situation in which the number of variables, impact, length, and a direction of conversation is not known. For example, if Julia, a Serenity chatbot user (or a patient), messages the conversational agent or chatbot in an unstructured conversation based therapeutic module, we may know the clinical prompts such as “What thoughts do you have”, but the patient's response to the prompt is unknown in advance. Julia can say “I am thinking about walking my dog in Richmond Hill later”, or she can provide any number of responses, ranging from irrelevant information to self-harm.

The technology disclosed can handle digressions and tangential conversations as a result of unstructured conversations with patients. A therapist can handle such situations naturally however, it is a challenge for chatbot to detect patient's intent to shift topics and respond appropriately. The technology disclosed can handle unstructured conversations when chatting with the patients by firstly understanding the contents of the patient's utterance. Secondly, when the content is understood and co-references are resolved, the technology disclosed can understand the meaning and desire of the content which is referred to as intent detection. Thirdly, based on the detected intent, the chatbot presents appropriate responses or treatment options to the patient (fulfillment). The illustration 832 indicates the three steps presented above in the form of overlapping ovals. Understanding the utterance and intent detection can be considered as part of natural language processing (NLP). The fulfillment is based on the skills of the therapist and our technology enables the chatbot to provide high quality therapy experience to patients.

For example, consider Serenity chatbot asks Julia, the patient, “How are you feeling?”. Julia replies “I'm doing great” (utterance). Serenity interprets her reply to understand she is feeling positive (intent), and responds “That is great to hear!” (fulfillment). Understanding individual statements and sentiments is the first step in providing a superior conversational experience. The next step is taking that into account when dealing with the conversational structure between two individuals, and the many tangential pathways it can take. The technology disclosed includes the logic to handle tangential conversations. We present examples of tangential conversations in the following sections.

### Tangential Conversations Examples

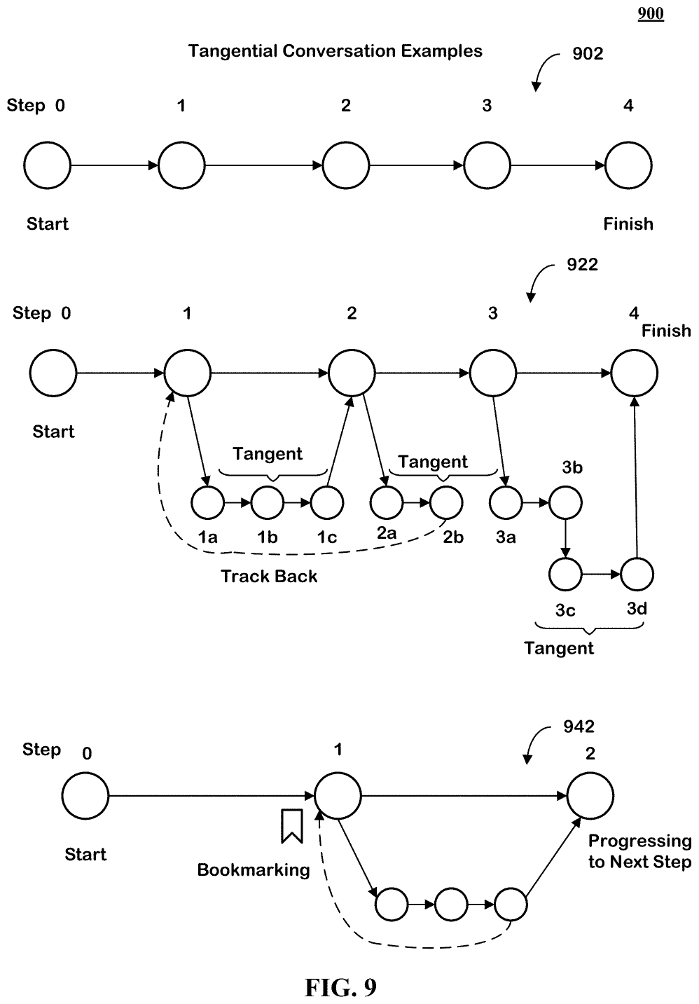

FIGS. 9, 10 and 11 present examples of tangential conversations. The examples illustrate various scenarios in which the chatbot can deal with non-linear conversational flow, tangents and add bookmarks to resume the CBT therapy session after completing the tangential conversation.

FIG. 9 shows abstract state machines for three example conversations of a patient with the mental health chatbot. The first conversation labeled 902 is a linear conversation and can be considered as an example of a structured conversation mentioned above. Conversations that limit the variability in the input a user can provide are likely to have their path as more definite and defined. In such a conversation no matter which option a patient chooses, she seamlessly moves from step 1 to step 4 via intermediate steps and completes the defined pathway of the conversation. An example of this might be a multiple-choice survey. If Julia, the patient is provided a survey, asking on a scale of 1 to 3, for ten questions, how she might rate her experience she is attending, there are a limited number of ways the flow of her conversation can go. No matter what she chooses, the conversation progresses to the next step of the survey.

The above example, however, is not how humans communicate with other humans, and is ineffective in delivering impactful mental health therapy sessions. Humans often do not follow in a linear pattern when conversing. They often veer off into tangents, loop back to old parts of the conversation, and introduce a variety of variables that are very hard to account for. The conversation 922 in FIG. 9 is an example of conversation with tangential paths. The conversation starts at step 0 and finishes at step 4. There are three branches between steps 1 and 2, steps 2 and back to 1, and between steps 3 and 4. The first branch has steps 1a, 1b, 1c. The second branch has steps 2a, 2b and then track backs to step 1. The third branch has steps 3a, 3b, 3c, and 3d.

A conversation with branches can occur in the following example scenario. Suppose the therapy chatbot asks the patient Julia “How are you doing?”. This might have been one of many questions the inquirer had in mind when conversing with Julia, expecting a short answer, and moving onto the next part of the sequence in the therapy session. But the response of patient cannot be predicted by the therapy chatbot. Julia might go into a long story, discussing how she was walking her dog, Kuba, in Richmond Hill, but then it was interrupted by a call from her boss asking about the status of the latest financial models. She can then go off on a tangent based upon a previous part of the sequence, or a prior session, in which she discussed her boss and this creates a new branch in the flow. She might go so far down this tangential story, that she loops back to the original question and place in the original sequence, in order to return to the conversational path.

In order to account for this deviation in conversations, and ability to return to the predefined sequence, the technology disclosed includes a concept called “Bookmarking”. In the bookmarking technique, the chatbot makes note of when and where a patient goes off the predefined path in our abstract state machine. When going down a tangential conversational branch with the patient the chatbot has a clear last state to return to in the sequence via the defined bookmark when the tangential conversation reaches a conclusion. Tangential conversations can leverage bookmarking to return back to the last defined node in the sequence, or progress to the next portion of the conversation. The conversation 942 in FIG. 9 shows a bookmark at step 1 in the conversation from where the tangential conversation started. When the tangential conversation ends, the system can resume the main CBT therapy session from a next step (step 2) after the last completed step (step 1). We now present more detailed examples of how the system can use artificial memory and tangential conversation technique to provide a therapy experience to patients that feels like working with a CBT therapist.

### Example of Using Artificial Memory and Tangential Conversation

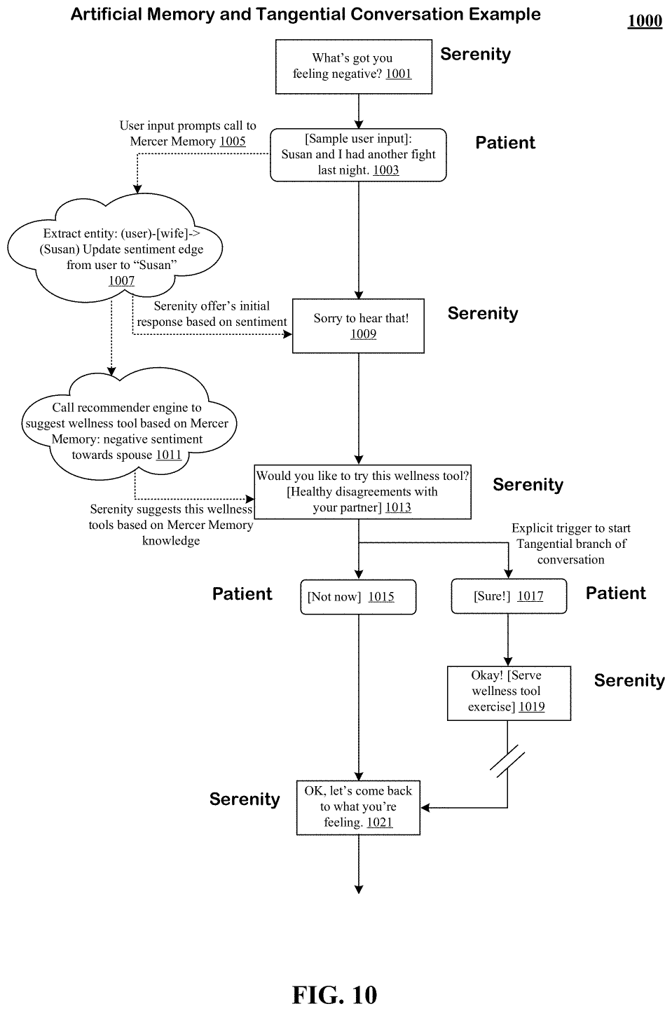

FIG. 10 presents an example conversation in which the chatbot queries artificial memory to extract relationship of an entity mentioned in the utterance from the patient. The example also includes invitation from the chatbot to start a tangential conversation. The conversation presented below can be part of a CBT therapy session, we are presenting the part of the conversation in the therapy relevant for illustration purposes. The Serenity mental health chatbot asks the user “What's got you feeling negative?” (1001). The patient responds with an utterance, “Susan and I had another fight last night” (1003). The patient's input is used to generate a query to the artificial memory (1005). The query (user)-[wife]->(Susan) extracts the relationship “wife” of the entity “Susan” mentioned in the utterance of the patient. The system updates the sentiment edge connecting the patient root node to the entity node representing his wife with a label that indicates negative sentiment of the patient towards his wife (1007). The chatbot offers an initial response to the patient based on the sentiment of the patient towards the entity, “Sorry to hear that!” (1009).

The system can use a recommender engine to suggest wellness tool or a therapy exercise to the patient based on negative sentiment of the patient towards spouse (1011). The chatbot sends a prompt to the user, “Would you like to try this wellness tool? [Healthy disagreements with your partner]” (1013). The patient can respond “Not now” and reject the invitation (1015). If the patient accepts chatbot's invitation and utters something like “Sure!” as shown in the box 1017, the system can start a tangential conversation. The above invitation from the chatbot in box 1013 is an explicit trigger to start the tangential branch. The chatbot sends a prompt “Okay!” to the patient and serves the well tool exercise regarding healthy disagreements with spouse (1019). After completing the tangential branch, the chatbot returns back to the main branch and sends a prompt to the patient “Ok, let's come back to what you're feeling” (1021).

### Example of Suggesting Multiple Wellness Tools to the Patient

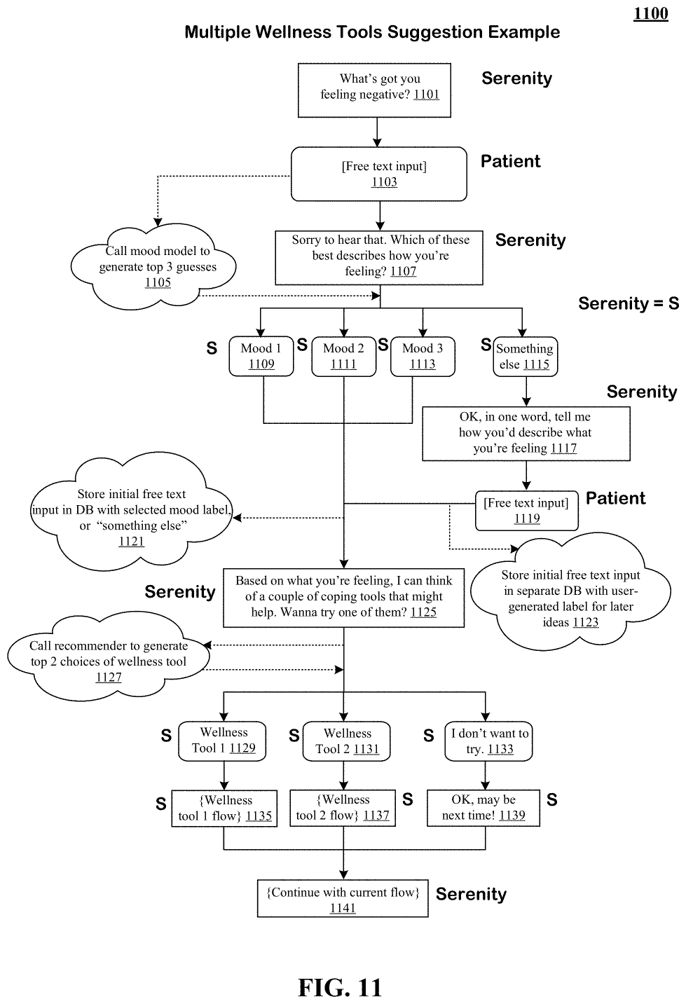

In this example, the chatbot suggests multiple wellness tools to the patient based on patient's utterance. The conversation shown in FIG. 11 can be part of a therapy session. We are showing only a part of the conversation for illustration purposes. The Serenity mental health chatbot asks the user “What's got you feeling negative?” (1101). The patient responds with an utterance 1103. The patient's input is used by a mood model to generate top three guesses about what the patient is feeling (1105). The chatbot sends a prompt to the patient “Sorry to hear that. Which of these best describe how you're feeling?” (1107). The chatbot then presents the top three mood guesses from the mood model: mood 1 (1109), mood 2 (1111), mood 3 (1113) to the patient. The chatbot also presents a “something else” option 1115 to the patient. The patient can select any one of the three moods using input buttons on the user interface. The patient can also select “something else” option if none of the suggested three moods describe what the patient is feeling. If the patient selects this option, the chatbot can ask the patient “OK, in one word, tell me how you′d describe what you're feeling” (1117). The patient can respond by describing what she is feeling (1119).

The system can store the selected option (from three mood options) from the patient describing her mood in a database. If the patient selects the “something else” option, the system can store the mood description provided by the patient (1121). The chatbot responds to the patient as, “Based on what you're feeling, I can think of a couple of coping tools that might help. Wanna try one of them?” (1125). The system can use the recommender engine to generate top 2 or more choices of wellness tools (1127). The patient can select well tool 1 (1129), wellness tool 2 (1131) or the patient can select the option “I don't want to try” (1133). The system will start the conversation flow for the selected wellness tool 1135, or 1137. If the patient does not want to try a tool, the chatbot can send a prompt “OK, may be next time!” (1139) to the patient. At the end of wellness tool flow, the system can resume the main conversation path of the therapy session (1141).

### Particular Implementations

We describe various implementations of chatbot technology in the context of a counseling oriented application useful for cognitive behavioral therapy. Of course, the technology has many additional applications.

The technology disclosed can be practiced as a system, method, or article of manufacture. One or more features of an implementation can be combined with the base implementation. Implementations that are not mutually exclusive are taught to be combinable. One or more features of an implementation can be combined with other implementations. This disclosure periodically reminds the user of these options. Omission from some implementations of recitations that repeat these options should not be taken as limiting the combinations taught in the preceding sections—these recitations are hereby incorporated forward by reference into each of the following implementations.

A method implementation of a portion of the technology disclosed provides an artificial memory useful for remembering content between and during conversations with a particular individual, including therapy sessions with a patient. The method technology can be applied to remembering for future use content received from a patient and understood by computerized natural language processing during a chatbot therapy session. This method includes maintaining a graph of entities and relationships in an artificial memory graph data structure. The entity nodes represent entities that are related to a patient root node. This root node represents the patient. One or more relationship edges connected to the root node that have named roles. The entity nodes and/or relationship edges are slotted for at least a distinguishing name, a patient sentiment towards the entity and an entity state. The method also includes maintaining a relationship hierarchy that describes alternative relationship edges used to connect the patient root node to a particular entity node This the relationship hierarchy is structured as hypernym and hyponym relationships. The method involves processing utterances from the patient in the chatbot therapy sessions. The utterances can address some or all of relationships and entities, sentiments of the patient towards the entities, and states of the entities. The technology disclosed repeatedly updates entity nodes and/or relationship edges using a relationship, a distinguishing name, or both found in particular utterances.

In various implementations, four or more types of utterances and updating can be apply. For first particular utterances, the method can involve querying for both the distinguishing name and the relationship and, when both are found, using sentiment and state information from the first particular utterances to fill or update one or more slots in the entity node and/or relationship edge to which the sentiment and state information relate.

For second particular utterances, the method can involve querying for the distinguishing name and, when a responsive named entity node is found, using information from the second particular utterances to fill or update one or more slots for the named entity node or the relationship edge connected to the named entity node to the patient root.

For third particular utterances, the method can involve querying for the relationship and, when a responsive relationship edge is found, using information from the third particular utterances to fill or update one or more slots for the relationship edge or the entity node related to the patient by the edge; and

For fourth particular utterances, when neither the distinguishing name nor the relationship are found by querying, the method can involve creating a new entity node and a new relationship edge using information from the fourth particular utterances and further filling in from the utterances one or more slots in each of the new entity node and the new relationship edge.

All four types of utterances can be combined in a method that practices the technology disclosed.

These method implementations and other methods disclosed can be used with one or more of the following features. This method can also include features described in connection with systems disclosed. In the interest of conciseness, alternative combinations of method features are not individually enumerated. Features applicable to methods, systems, and articles of manufacture are not repeated for each statutory class set of base features. The reader will understand how features identified in this section can readily be combined with base features in other statutory classes.

The technology disclosed can resolve among multiple edges found when querying for a relationship. For third particular utterances, the method can further involve querying for the relationship and, when multiple edges are found, extracting distinguishing names for multiple entity nodes connected by the multiple edges to the node representing the patient. Using information from the third particular utterance, the method includes sending a prompt to the patient using the sentiment of the patient towards the entities represented by the multiple entity nodes and a prompting the patient to select an entity from the multiple entities extracted from the artificial memory graph. A selected distinguishing name of the entity can be used in the following utterance to select the entity node in the artificial graph, and fill or update the patient sentiment towards the entity node.

Another method implementation of part of the technology disclosed makes use of remembered content, such as content collected by the method above. This is a method of making use of remembered content from earlier interactions with a patient in a series of sessions, during a therapy session. Doing so can include processing input from a patient in a particular utterance in a current conversation session. The input can include an entity having a distinguishing name and a relationship to the patient, which may be unstated, and an event involving the entity. The method includes using the event to infer a sentiment of the patient in the particular utterance towards the entity. It also involves accessing an artificial memory graph data structure comprising a patient root and entity nodes connected to the patient root by relationship edges, using the distinguishing name from the particular utterance. This accessing can include, for at least some forms of database, querying for the distinguishing name to select a named entity node, and extracting a role from at least one relationship edge between the patient root and the named entity node. Then, referring, in a prompt to the patient, to the named entity using the extracted role and the inferred sentiment to advance the conversation. This method can be used with one or more of the following features.

The method can further include advancing the conversation by proposing a therapy exercise based on the inferred sentiment and extracted relationship. It also can include updating, in the artificial memory, a sentiment of the patient towards the named entity node based on the particular utterance.

Another method implementation handles multiple entities found using relationship. This is a method of processing input from a patient in a particular utterance in a current conversation session. The input can include a named relationship and an event involving an entity. Actions include using the event to infer a sentiment of the patient in the particular utterance towards the entity. Actions also include accessing an artificial memory graph data structure comprising a patient root and entity nodes connected to the patient root by relationship edges, using the named relationship from the particular utterance. This access can include querying for the named relationship, selecting relationship edges connected to the patient root that are responsive to the query, and extracting a plurality of distinguishing names from entity nodes connected to the patient by the selected relationship edges. Then, sending a prompt to the patient asking the patient to select among the distinguishing names to disambiguate the named relationship and connected entity. The method advances the conversation by referring to the distinguishing name of the disambiguated entity and the inferred sentiment. This method can be used with one or more of the following features.

As above, the method can further include updating, in the artificial memory, a sentiment of the patient towards the disambiguated entity based on the particular utterance. It can include receiving, in response to the prompt, a distinguishing name for a new entity in a following utterance from the patient to the prompt. Further, updating the artificial memory by adding a new entity node representing the new entity and updating the new entity node by filling slots with the distinguishing name for the new entity and the sentiment of the patient towards the new entity node based on the particular utterance and connecting the new entity node with the patient root by a relationship edge with the named relationship. Then, referring to the distinguishing name of the new entity and the inferred sentiment during the conversation.

The method can be extended by checking in with the patient, asking about sentiment of the patient regarding an entity from a previous conversation. This can include sending a check-in prompt to the patient in a conversation session following the current conversation session, the prompt including a request for status update for a particular entity related to the patient with a negative sentiment from the patient towards the particular entity. In a response utterance from the patient, it includes a new positive sentiment of the patient towards the particular entity, followed by updating, in the artificial memory, the positive sentiment of the patient towards the particular entity in the entity node representing the particular entity. The method advances the conversation using the distinguishing name and the positive sentiment of the patient towards the particular entity.

Practicing the method, the entity node for the connected entity can include a time stamped attribute (such as born on.) It involves querying for the named relationship, further including, extracting a plurality of time stamped attributes from entity nodes connected to the patient by the selected relationship edges. And, comparing the time stamps in the time stamped attributes with a threshold and selecting the connected entity when the time stamp of the attribute from the entity node is less than the threshold. This extension advances the conversation using the inferred sentiment and the named relationship to propose a therapy exercise (or coping tool) to the patient.

The method can be further extended by processing input from a patient in a particular utterance in a current conversation session, the input including a state for the entity involved. The actions involve querying for the named relationship, extracting a state for the entity node connected to the patient by the relationship edge, updating the extracted state of the entity node connected to the patient with the new state (such as dead) for the connected entity. And, referring to the connected entity using the distinguishing name, the new state of the connected entity and the inferred sentiment to advance the conversation.

The method also can be extended by processing input from the patient further including a new workplace identifier related to the entity involved. The actions involve querying for the named relationship, extracting a workplace identifier for the entity node connected to the patient by the relationship edge, updating the extracted workplace identifier for the entity node connected to the patient with the new workplace identifier for the connected entity. And, referring to the connected entity using the distinguishing name, and the inferred sentiment to advance the conversation.

Another method that implements aspects of the technology disclosed handles tangential conversations. This implementation addresses detecting when to diverge to a tangential conversation from linearly structured steps of a cognitive behavioral therapy (CBT) session in a series of sessions for a mental health disorder and when to merge back to flow of linearly structured steps of the session from the tangential conversation in the therapy session. The method includes conducting a conversation in a current CBT therapy session in the series of sessions for the disorder between a chatbot and a patient. A computer-implemented system receives input from the patient in a particular utterance in the conversation and detects a present intent of the patient to diverge from the flow of linearly structured steps of the CBT session. Then it sends an invitation to the patient to diverge from the flow of the CBT session and start a tangential conversation.

In some implementations, the method continues with receiving a confirmation from the patient to start the tangential conversation and bookmarking a last completed step in the flow of linearly structured steps of the CBT therapy session. Then, starting the tangential conversation. As the conversation proceeds, the system calculates a return to CBT session score after each succeeding utterance from the patient and compares the return to CBT session score with a threshold. Upon detecting that the CBT session score has exceeded the threshold, it resumes the flow of the linearly structured steps of the CBT session from a step following the bookmarked last completed. This method can be used with one or more of the following features.

This method can be applied, for instance, responsive to the utterance “my cat passed away.” The method can apply in a series of sessions between a chatbot and a patient directed to treating a negative thoughts mental health disorder, or more generally. The action of sending the invitation to the patient to diverge from the flow of the CBT session and start the tangential conversation can include processing input from the patient in the particular utterance in the CBT session. The input can include a relationship, which is represented by a relationship edge connecting the patient to an entity represented as an entity node and having a distinguishing name. An implicit role can be determined based on the relationship edge. The input can further include a personal loss event including the entity, such as my cat passed away. The method includes accessing, an artificial memory graph data structure, comprising the entity nodes connected by relationship edges, using the implicit role determined by the relationship edge, when an entity is found, extracting the distinguishable name of the entity from the entity node, and updating the entity node with a sentiment (such as sadness) of the patient towards the entity inferred from the event and a state of the entity (such as dead.) Then, sending the invitation to the patient further including, using the patient sentiment towards the entity and the distinguishable name of the entity to recommend a personal loss therapy exercise for the patient in the invitation.

This method also can be applied to an utterance such as, “Susan and I had a fight last night.” Susan's name can be used to extract a wife relationship. This can apply in a series of sessions between a chatbot and a patient directed to treating a negative thoughts mental health disorder, or more generally. The action of processing input from the patient in the particular utterance in the current conversation session, can include receiving input. The input can include an entity related to the patient, represented as an entity node, having a distinguishing entity name, and having an implicit role determined by a relationship edge. It also can include a domestic or workplace or conflict event including the entity, such as a spouse or a coworker for workplace conflict. The method can further include accessing an artificial memory graph data structure, comprising the entity nodes connected by relationship edges, using the distinguishing name. When an entity is found, extracting the implicit role determined by the relationship edge connecting the node representing the patient to the entity node. Then updating the entity node with a sentiment (e.g., anger) of the patient towards the entity inferred from the event.

In some implementations, the method further includes sending the invitation to the patient. This can include using the patient sentiment towards the entity and the implicit role determined by the relationship edge to recommend a therapy exercise for the patient in the invitation.

Practicing this method can include inferring an anxiety sentiment from the personal event and sending the invitation to the patient using the anxiety sentiment of the patient to send a prompt to elicit further information about the personal event for use in selection of a therapy exercise (or coping module or other anxiety-related module) for the patient.

The method can further include, when calculating the return to CBT session score, comparing a character count in a current utterance in the tangential conversation between the chatbot and the patient to a character count in a preceding utterance in the tangential conversation. The result of the comparing can be used to increment incrementing the return to CBT session score when the character count in the current utterance is less than or equal to the character count in the preceding utterance.

Similarly, this method can further include, when calculating the return to CBT session score, incrementing the return to CBT session score when the sentiment of the patient in the current utterance indicates that the patient's interest in the conversation is waning.

Further, this method can further include, when calculating the return to CBT session score, processing the input in a current utterance in the tangential conversation between the chatbot and the patient to determine a personal event related to the patient and incrementing the return to CBT session score to set a value above the threshold to return to the CBT session. Then, sending an invitation to the patient, using a sentiment of the patient inferred from the personal event to elicit whether the patient wants to start a new tangential conversation for the personal event or resume the flow of the CBT session.

When calculating the return to CBT session score, the method can detecting in the patient's utterance an indication to end the tangential conversation and sending a prompt to the patient including an indication that chatbot is resuming the flow of the CBT session from a step following the bookmarked step in the linearly structured steps of the CBT session.

Calculating the return to CBT session score can also include counting utterances in the tangential conversation between the chatbot and the patient and incrementing the return to CBT session score as the count of utterances increases above a threshold for a recommended number of utterances in the tangential conversation.

Each of the features discussed in this particular implementation section for the one method implementation apply equally to all other method implementation, except where incompatible. As indicated above, all the method features are not repeated in second and subsequent methods and should be considered repeated by reference.

System implementations of the technology disclosed include one or more processors coupled to memory. The memory is loaded with computer instructions executable by the processors to carry out any of the methods above, including combinations of methods with one or more features, extensions or options.

Other implementations may include a non-transitory computer readable storage medium storing instructions executable by a processor to perform functions of the system described above. Yet another implementation may include a method performing the functions of the system described above.

Other implementations may include a non-transitory computer readable storage medium storing instructions executable by a processor to perform a method as described above. Yet another implementation may include a non-transitory computer readable storage medium storing instructions executable by a processor that, when combined with suitable hardware, produce any of the systems described herein.

Reference to computer readable medium is not intended to refer to transitory signals carrying computer instructions executable by a processor, which are within the scope of the technology disclosed. When transitory signals are deemed patentable, they will be claimed as signals or downloads or some other form of data in motion, as opposed to data at rest.

### Computer System

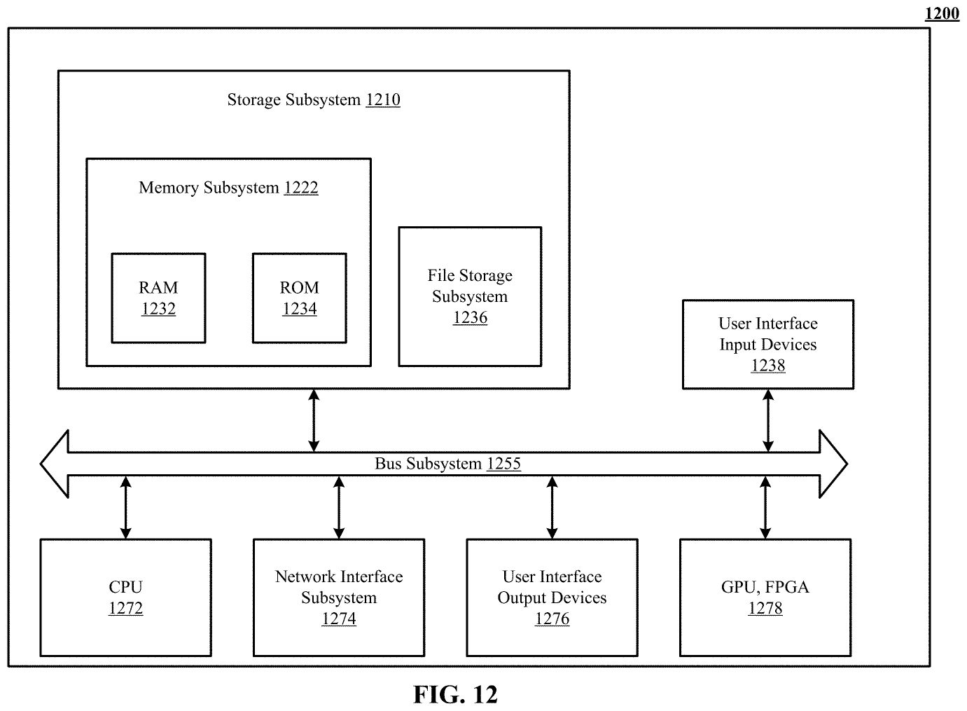

FIG. 12 is a simplified block diagram of a computer system 1200 that can be used to implement the technology disclosed. Computer system typically includes at least one processor 1272 that communicates with a number of peripheral devices via bus subsystem 1255. These peripheral devices can include a storage subsystem 1210 including, for example, memory subsystem 1222 and a file storage subsystem 1236, user interface input devices 1238, user interface output devices 1276, and a network interface subsystem 1274. The input and output devices allow user interaction with computer system. Network interface subsystem provides an interface to outside networks, including an interface to corresponding interface devices in other computer systems.

In one implementation, the mental health chatbot 133 is communicably linked to the storage subsystem and user interface input devices.

User interface input devices 1238 can include a keyboard; pointing devices such as a mouse, trackball, touchpad, or graphics tablet; a scanner; a touch screen incorporated into the display; audio input devices such as voice recognition systems and microphones; and other types of input devices. In general, use of the term “input device” is intended to include all possible types of devices and ways to input information into computer system.

User interface output devices 1276 can include a display subsystem, a printer, a fax machine, or non-visual displays such as audio output devices. The display subsystem can include a cathode ray tube (CRT), a flat-panel device such as a liquid crystal display (LCD), a projection device, or some other mechanism for creating a visible image. The display subsystem can also provide a non-visual display such as audio output devices. In general, use of the term “output device” is intended to include all possible types of devices and ways to output information from computer system to the user or to another machine or computer system.

Storage subsystem 1210 stores programming and data constructs that provide the functionality of some or all of the modules and methods described herein. These software modules are generally executed by processor alone or in combination with other processors.

Memory used in the storage subsystem can include a number of memories including a main random access memory (RAM) 1232 for storage of instructions and data during program execution and a read only memory (ROM) 1234 in which fixed instructions are stored. The file storage subsystem 1236 can provide persistent storage for program and data files, and can include a hard disk drive, a floppy disk drive along with associated removable media, a CD-ROM drive, an optical drive, or removable media cartridges. The modules implementing the functionality of certain implementations can be stored by file storage subsystem in the storage subsystem, or in other machines accessible by the processor.

Bus subsystem 1255 provides a mechanism for letting the various components and subsystems of computer system communicate with each other as intended. Although bus subsystem is shown schematically as a single bus, alternative implementations of the bus subsystem can use multiple busses.

Computer system itself can be of varying types including a personal computer, a portable computer, a workstation, a computer terminal, a network computer, a television, a mainframe, a server farm, a widely-distributed set of loosely networked computers, or any other data processing system or user device. Due to the ever-changing nature of computers and networks, the description of computer system depicted in FIG. 12 is intended only as a specific example for purposes of illustrating the technology disclosed. Many other configurations of computer system are possible having more or less components than the computer system depicted in FIG. 12.

The computer system 1200 includes GPUs or FPGAs 1278. It can also include machine learning processors hosted by machine learning cloud platforms such as Google Cloud Platform, Xilinx, and Cirrascale. Examples of deep learning processors include Google's Tensor Processing Unit (TPU), rackmount solutions like GX4 Rackmount Series, GX8 Rackmount Series, NVIDIA DGX-1, Microsoft' Stratix V FPGA, Graphcore's Intelligent Processor Unit (IPU), Qualcomm's Zeroth platform with Snapdragon processors, NVIDIA's Volta, NVIDIA's DRIVE PX, NVIDIA's JETSON TX1/TX2 MODULE, Intel's Nirvana, Movidius VPU, Fujitsu DPI, ARM's DynamicIQ, IBM TrueNorth, and others.

We claim as follows:

1. A method of remembering for future use content received from a patient and understood by computerized natural language processing during a chatbot therapy session, including:
maintaining a graph of entities and relationships in an artificial memory graph data structure, wherein entity nodes represent entities that are related to a patient root node representing the patient by one or more relationship edges that have named roles, wherein the entity nodes and/or relationship edges are slotted for at least a distinguishing name, a patient sentiment towards the entity and an entity state;
maintaining a relationship hierarchy that describes alternative relationship edges used to connect the patient root node to a particular entity node, wherein the relationship hierarchy is structured as hypernym and hyponym relationships;
processing utterances from the patient in the chatbot therapy sessions, the utterances including:
relationships and entities;
sentiments of the patient towards the entities, and
states of the entities;
repeatedly updating entity nodes and/or relationship edges using a relationship, a distinguishing name, or both found in particular utterances, including:
for first particular utterances, querying for both the distinguishing name and the relationship and, when both are found, using sentiment and state information from the first particular utterances to fill or update one or more slots in the entity node and/or relationship edge to which the sentiment and state information relate,
for second particular utterances, querying for the distinguishing name and, when a responsive named entity node is found, using information from the second particular utterances to fill or update one or more slots for the named entity node or the relationship edge connected to the named entity node to the patient root,
for third particular utterances, querying for the relationship and, when a responsive relationship edge is found, using information from the third particular utterances to fill or update one or more slots for the relationship edge or the entity node related to the patient by the edge; and
for fourth particular utterances, when neither the distinguishing name nor the relationship are found by querying, creating a new entity node and a new relationship edge using information from the fourth particular utterances and further filling in from the utterances one or more slots in each of the new entity node and the new relationship edge.

2. The method of claim 1, further including:
for third particular utterances, querying for the relationship and, when multiple edges are found, extracting distinguishing names for multiple entity nodes connected by the multiple edges to the node representing the patient, using the information from the third particular utterances to send a prompt to the patient using the sentiment of the patient towards the entities represented by the multiple entity nodes and a prompt to the patient to select an entity from the multiple entities extracted from the artificial memory graph,
using the distinguishing name of the entity selected by the patient in the following utterance to select the entity node in the artificial memory graph, and fill in or update the patient sentiment towards the entity node.

3. A non-transitory computer readable storage medium impressed with computer program instructions to make use of remembered content during a therapy session from earlier interactions with a patient in a series of sessions, the instructions, when executed on a processor, implement a method comprising:
processing input from a patient in a particular utterance in a current conversation session, the input including:
an entity having a distinguishing name and a relationship to the patient, which may be unstated, and
an event involving the entity;
using the event to infer a sentiment of the patient in the particular utterance towards the entity;
accessing an artificial memory graph data structure comprising a patient root and entity nodes connected to the patient root by relationship edges, using the distinguishing name from the particular utterance, including:
querying for the distinguishing name to select a named entity node, and
extracting a role from at least one relationship edge between the patient root and the named entity node, and
referring, in a prompt to the patient, to the named entity using the extracted role and the inferred sentiment to advance the conversation.

4. The non-transitory computer readable storage medium of claim 3, implementing the method further comprising:
advancing the conversation by proposing a therapy exercise based on the inferred sentiment and extracted relationship.

5. The non-transitory computer readable storage medium of claim 3, implementing the method further comprising:
updating, in the artificial memory, a sentiment of the patient towards the named entity node based on the particular utterance.

6. A system including one or more processors coupled to memory, the memory loaded with computer instructions to make use of remembered content during a therapy session from earlier interactions with a patient in a series of sessions, when executed on the processors implement the instructions of claim 3.

7. The system of claim 6, further implementing actions comprising:
advancing the conversation by proposing a therapy exercise based on the inferred sentiment and extracted relationship.

8. The system of claim 6, further implementing actions comprising:
updating, in the artificial memory, a sentiment of the patient towards the named entity node based on the particular utterance.

9. A non-transitory computer readable storage medium impressed with computer program instructions to process input from a patient in a particular utterance in a current conversation session, the instructions, when executed on a processor, implement a method comprising, the input including:
a named relationship, and
an event involving an entity,
using the event to infer a sentiment of the patient in the particular utterance towards the entity;
accessing, an artificial memory graph data structure comprising a patient root and entity nodes connected to the patient root by relationship edges, using the named relationship from the particular utterance, including:
querying for the named relationship, selecting relationship edges connected to the patient root that are responsive to the query, and extracting a plurality of distinguishing names from entity nodes connected to the patient by the selected relationship edges, and
sending a prompt to the patient asking the patient to select among the distinguishing names to disambiguate the named relationship and connected entity,
advancing the conversation by referring to the distinguishing name of the disambiguated entity and the inferred sentiment.

10. The non-transitory computer readable storage medium of claim 9, implementing the method further comprising:
updating, in the artificial memory, a sentiment of the patient towards the disambiguated entity based on the particular utterance.

11. The non-transitory computer readable storage medium of claim 9, implementing the method further comprising:
receiving, in response to the prompt, a distinguishing name for a new entity in a following utterance from the patient to the prompt;
updating, the artificial memory by adding a new entity node representing the new entity and updating the new entity node by filling slots with the distinguishing name for the new entity and the sentiment of the patient towards the new entity node based on the particular utterance, connecting the new entity node with the patient root by a relationship edge with the named relationship;
advancing the conversation by referring to the distinguishing name of the new entity and the inferred sentiment.

12. The non-transitory computer readable storage medium of claim 9, implementing the method further comprising:
sending a check-in prompt to the patient in a conversation session following the current conversation session, the prompt including a request for status update for a particular entity related to the patient with a negative sentiment from the patient towards the particular entity;
receiving, in a response utterance from the patient, a new positive sentiment of the patient towards the particular entity;
updating, in the artificial memory, the positive sentiment of the patient towards the particular entity in the entity node representing the particular entity;
advancing the conversation using the distinguishing name and the positive sentiment of the patient towards the particular entity.

13. The non-transitory computer readable storage medium of claim 9, wherein the entity node for the connected entity includes a time stamped attribute (such as born on), the method further including:
querying for the named relationship, further including, extracting a plurality of time stamped attributes from entity nodes connected to the patient by the selected relationship edges, and
comparing the time stamps in the time stamped attributes with a threshold and selecting the connected entity when the time stamp of the attribute from the entity node is less than the threshold,
further including, advancing the conversation using the inferred sentiment and the named relationship to propose a therapy exercise (or coping tool) to the patient.

14. The non-transitory computer readable storage medium of claim 9, implementing the method further comprising:
processing input from a patient in a particular utterance in a current conversation session, the input including a state for the entity involved,
querying for the named relationship, extracting a state for the entity node connected to the patient by the relationship edge, updating the extracted state of the entity node connected to the patient with a new state (such as dead) for the connected entity,
referring to the connected entity using the distinguishing name, the new state of the connected entity and the inferred sentiment to advance the conversation.

15. The non-transitory computer readable storage medium of claim 9, implementing the method further comprising:
processing input from the patient further including a new workplace identifier related to the entity involved,
querying for the named relationship, extracting a workplace identifier for the entity node connected to the patient by the relationship edge, updating the extracted workplace identifier for the entity node connected to the patient with the new workplace identifier for the connected entity,
referring to the connected entity using the distinguishing name, and the inferred sentiment to advance the conversation.

16. A system including one or more processors coupled to memory, the memory loaded with computer instructions to process input from a patient in a particular utterance in a current conversation session, when executed on the processors implement the instructions of claim 9.

17. The system of claim 16, further implementing actions comprising:
updating, in the artificial memory, a sentiment of the patient towards the disambiguated entity based on the particular utterance.

18. The system of claim 16, further implementing actions comprising:
receiving, in response to the prompt, a distinguishing name for a new entity in a following utterance from the patient to the prompt;
updating, the artificial memory by adding a new entity node representing the new entity and updating the new entity node by filling slots with the distinguishing name for the new entity and the sentiment of the patient towards the new entity node based on the particular utterance, connecting the new entity node with the patient root by a relationship edge with the named relationship;
advancing the conversation by referring to the distinguishing name of the new entity and the inferred sentiment.

19. The system of claim 16, further implementing actions comprising:
sending a check-in prompt to the patient in a conversation session following the current conversation session, the prompt including a request for status update for a particular entity related to the patient with a negative sentiment from the patient towards the particular entity;
receiving, in a response utterance from the patient, a new positive sentiment of the patient towards the particular entity;
updating, in the artificial memory, the positive sentiment of the patient towards the particular entity in the entity node representing the particular entity;
advancing the conversation using the distinguishing name and the positive sentiment of the patient towards the particular entity.

20. The system of claim 16, wherein the entity node for the connected entity includes a time stamped attribute (such as born on), further implementing actions comprising:
querying for the named relationship, further including, extracting a plurality of time stamped attributes from entity nodes connected to the patient by the selected relationship edges, and
comparing the time stamps in the time stamped attributes with a threshold and selecting the connected entity when the time stamp of the attribute from the entity node is less than the threshold,
further including, advancing the conversation using the inferred sentiment and the named relationship to propose a therapy exercise (or coping tool) to the patient.

21. The system of claim 16, further implementing actions comprising:
processing input from a patient in a particular utterance in a current conversation session, the input including a state for the entity involved,
querying for the named relationship, extracting a state for the entity node connected to the patient by the relationship edge, updating the extracted state of the entity node connected to the patient with a new state (such as dead) for the connected entity,
referring to the connected entity using the distinguishing name, the new state of the connected entity and the inferred sentiment to advance the conversation.

22. The system of claim 16, further implementing actions comprising:
processing input from the patient further including a new workplace identifier related to the entity involved,
querying for the named relationship, extracting a workplace identifier for the entity node connected to the patient by the relationship edge, updating the extracted workplace identifier for the entity node connected to the patient with the new workplace identifier for the connected entity,
referring to the connected entity using the distinguishing name, and the inferred sentiment to advance the conversation.
